(19) United States
(12) Patent Application Publication
(10) Pub. No.: US 2017/0216305 A1
(43) Pub. Date: Aug. 3, 2017

(54) COMPOSITIONS COMPRISING MELOXICAM-CYCLODEXTRIN INCLUSION COMPLEXES AND METHODS OF TREATING ACUTE PAIN

(71) Applicants: Hong Sun, Shanghai (CN); Yan Hu, Shanghai (CN); Yansheng Chen, Shanghai (CN); Luwei Zhao, Shanghai (CN)

(72) Inventors: Hong Sun, Shanghai (CN); Yan Hu, Shanghai (CN); Yansheng Chen, Shanghai (CN); Luwei Zhao, Shanghai (CN)

(73) Assignee: Arissa Pharma Ltd., Shanghai (CN)

(21) Appl. No.: 15/515,211

(22) PCT Filed: Sep. 29, 2015
(86) PCT No.: PCT/IB2015/002670

§ 371 Date: Mar. 29, 2017

Related U.S. Application Data
(60) Provisional application No. 62/057,032, filed on Sep. 29, 2014.

(51) Int. Cl. A61K 31/5415; A61K 47/26

(52) U.S. Cl. CPC A61K 31/5415; A61K 47/26

(57) ABSTRACT

The present invention is directed to a method of administering a meloxicam formulation to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours.

### RELATED APPLICATIONS

This application claims the priority of U.S. provisional application Ser. No. 62/057,032, entitled “COMPOSITIONS COMPRISING MELOXICAM-CYCLODEXTRIN INCLUSION COMPLEXES AND METHODS OF TREATING ACUTE PAIN,” filed Sep. 29, 2014; which is incorporated herein by reference in its entirety for all purposes.

### FIELD OF THE INVENTION

The embodiments of the present invention relate to compositions comprising meloxicam-cyclodextrin inclusion complexes for treating mild to moderate acute pain and methods of use thereof.

### BACKGROUND

Meloxicam is a non-steroidal anti-inflammatory drug (NSAID) that exhibits anti-inflammatory, analgesic, and antipyretic activities. The active ingredient, meloxicam, is found in commercially available pharmaceutical formulations.

### SUMMARY OF THE INVENTION

In an embodiment, the present invention is directed to compositions including meloxicam and methods of treating pain and/or inflammation by administering the compositions to a subject in need.

In an embodiment, the present invention is a method of administering a meloxicam formulation to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours (e.g., but not limited to, 0.25 hours, 0.5 hours, 1 hour, 1.5 hours, 2 hours, 2.5 hours, 3.0 hours). In an embodiment, the oral solid dosage form further includes one or more pharmaceutically acceptable excipients. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the oral solid dosage form is selected from one of a capsule, a tablet, a sachet or granule powder. In an embodiment, the method of the present invention is for treating mild to moderate acute pain. In an embodiment, the mammalian subject is a human. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram (XRPD). In an embodiment, the amorphous meloxicam-cyclodextrin inclusion complex displays no characteristic endothermic peak of crystalline meloxicam at 250° C.-258° C. as evidenced by differential scanning calorimetry (DSC).

In an embodiment, the present invention is a method of administering meloxicam to a mammalian subject to manage mild to moderate acute pain in the subject including: orally administering to the subject an oral pharmaceutical formulation including an amorphous meloxicam-cyclodextrin inclusion complex, where upon administration in the subject and after an amount of meloxicam has been released from the formulation, is absorbed by the subject, and reaches the systemic circulation of the subject, a shorter Tₘₐₓ is achieved as compared with a standard commercial formulation of meloxicam (Tₘₐₓ is time to peak plasma concentration). In an embodiment, the shorter Tₘₐₓ is not greater than about 75% of the Tₘₐₓ exhibited by a standard commercial formulation of meloxicam. In an embodiment, the shorter Tₘₐₓ is not greater than about 50% of the Tₘₐₓ exhibited by a standard commercial formulation of meloxicam. In an embodiment, the shorter Tₘₐₓ is not greater than about 25% of the Tₘₐₓ exhibited by a standard commercial formulation of meloxicam. In an embodiment, when a lower strength (e.g., 5.5 mg) formulation of the present invention is administered to a person in need thereof, a lower Tₘₐₓ is achieved, but exhibits a comparable Cₘₐₓ (e.g., 80-125% C.) as compared with the standard oral commercial formulation of meloxicam (e.g., Mobic® 7.5 mg). In an embodiment, the formulation further includes one or more pharmaceutically acceptable excipients. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the formulation is selected from one of a capsule, a tablet, or a sachet or granule powder. In an embodiment, the mammalian subject is a human. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In an embodiment, the amorphous meloxicam-cyclodextrin inclusion complex displays no characteristic endothermic peak of crystalline meloxicam at 250° C.-258° C. as evidenced by differential scanning calorimetry.

In an embodiment, the present invention is a solid pharmaceutical formulation including an effective amount of meloxicam, and one or more pharmaceutically acceptable excipients, the formulation including an amorphous meloxicam-cyclodextrin inclusion complex sufficiently designed to provide a time of meloxicam peak plasma concentration (Tₘₐₓ) of not greater than about 2 hours after administration and a peak concentration (Cₘₐₓ) of meloxicam which is higher than Cₘₐₓ of a standard commercial formulation of meloxicam. In an embodiment, the pharmaceutical formulation provides effective pain control in a subject starting at about 15 minutes to 2 hours after administration and lasting for up to 24 hours after administration. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the formulation is selected from one of a capsule, a tablet, a sachet or granule powder. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In an embodiment, the amorphous meloxicam-cyclodextrin inclusion complex displays no characteristic endothermic peak of crystalline meloxicam at 250° C.-258° C. as evidenced by differential scanning calorimetry.

In an embodiment, the present invention is directed to a method of orally administering a meloxicam formulation to a mammalian subject for the treatment of mild to moderate acute pain and/or inflammation, with the formulation including a meloxicam-cyclodextrin inclusion complex. In an embodiment, after administration of the formulation, a fast onset of therapeutic effect in the subject is achieved. In an embodiment, the administration of the meloxicam formulation results in a shorter Tₘₐₓ as compared to Tₘₐₓ of a standard commercial oral formulation of meloxicam. In an embodiment, the therapeutic effect is to treat mild to moderate acute pain and/or inflammation. In an embodiment, the formulation produces a Tₘₐₓ not greater than about 75% of the Tₘₐₓ exhibited by a standard commercial oral formulation of meloxicam. In an embodiment, the formulation produces a Tₘₐₓ not greater than about 50% of the Tₘₐₓ exhibited by a standard commercial oral formulation of meloxicam. In an embodiment, the formulation produces a Tₘₐₓ not greater than about 25% of the Tₘₐₓ exhibited by a standard commercial oral formulation of meloxicam. In an embodiment, the strength of meloxicam in a formulation of the present invention is not greater than about 80% of the strength of meloxicam in a standard commercial oral formulation. In an embodiment, the strength of meloxicam in a formulation of the present invention is not greater than about 70% of the strength of meloxicam in a standard commercial oral formulation. In an embodiment, the strength of meloxicam in a formulation of the present invention is not greater than about 60% of the strength of meloxicam in a standard commercial oral formulation. In an embodiment, the meloxicam-cyclodextrin inclusion complex at a formulation of the present invention is prepared through a spray drying process. In an embodiment, meloxicam in the inclusion complex is in an amorphous state. In an embodiment, meloxicam in the inclusion complex displays typical amorphous halo and no characteristic peaks of crystalline meloxicam such as 13.1, 14.9, 18.6, 25.9° at 2θ scales, as evidenced by an X-ray powder diffractogram (XRPD). In an embodiment, meloxicam in the inclusion complex displays no characteristic endothermic peak of crystalline meloxicam at 250-258° C. as evidenced by differential scanning calorimetry (DSC). In an embodiment, the meloxicam formulation further includes pharmaceutically acceptable excipients. In an embodiment, the meloxicam formulation is in an oral dosage form of a capsule. In an embodiment, the meloxicam formulation is in an oral dosage form of a tablet. In an embodiment, the meloxicam formulation is in an oral dosage form of granule powder.

According to aspects illustrated herein, there is disclosed a method to administer a meloxicam formulation to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours (e.g., but not limited to, 0.25 hours, 0.5 hours, 1 hour, 1.5 hours, 2 hours, 2.5 hours, 3.0 hours). In some embodiments, the oral solid dosage form further includes one or more pharmaceutically acceptable excipients. In some embodiments, the cyclodextrin is β-cyclodextrin. In some embodiments, the cyclodextrin is a derivative of β-cyclodextrin. In some embodiment, the cyclodextrin is hydroxylpropyl-β-cyclodextrin. In some embodiments, the oral solid dosage form is a capsule. In some embodiments, the oral solid dosage form is a tablet. In some embodiments, the oral solid dosage form is granule powder. In some embodiments, the meloxicam formulation is used to treat mild to moderate acute pain and/or inflammation. In some embodiments, the mammalian subject is a human. In some embodiments, the amorphous meloxicam-cyclodextrin inclusion complex displays a typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

In an embodiment, the present invention is directed to a method to administer meloxicam to a mammalian subject to manage mild to moderate acute pain, and/or inflammation in the subject including: orally administering to the subject an oral pharmaceutical formulation including an amorphous meloxicam-cyclodextrin inclusion complex, where upon administration in the subject and after an amount of meloxicam has been released from the formulation, is absorbed by the subject, and reaches the systemic circulation of the subject, a shorter Tₘₐₓ is achieved as compared with a standard commercial oral formulation of meloxicam. In an embodiment, the shorter Tₘₐₓ is not greater than about 75% of the Tₘₐₓ exhibited by a standard commercial formulation of meloxicam. In some embodiments, the shorter Tₘₐₓ is not greater than about 50% of the Tₘₐₓ exhibited by a standard commercial oral formulation of meloxicam. In some embodiments, the shorter Tₘₐₓ is not greater than about 25% of the Tₘₐₓ exhibited by a standard commercial oral formulation of meloxicam. In some embodiments, upon administration in the subject and after an amount of meloxicam has been released from the formulation, meloxicam released from the formulation is absorbed by the subject, and reaches the systemic circulation of the subject, a higher Cₘₐₓ (peak plasma concentration) is achieved as compared with a standard commercial oral formulation of meloxicam. In some embodiments, the formulation further includes one or more pharmaceutically acceptable excipients. In some embodiments, the cyclodextrin is β-cyclodextrin. In some embodiments, the cyclodextrin is a derivative of β-cyclodextrin. In some embodiment, the cyclodextrin is hydroxylpropyl-β-cyclodextrin. In some embodiments, the formulation is selected from one of a capsule, a tablet or granule powder. In some embodiments, the mammalian subject is a human. In some embodiments, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

In some embodiments, the present invention is directed to a solid pharmaceutical formulation including an effective amount of meloxicam, and one or more pharmaceutically acceptable excipients, the formulation including an amorphous meloxicam-cyclodextrin inclusion complex sufficiently designed to provide a time of meloxicam peak plasma concentration (Tₘₐₓ) of not greater than about 3 hours after administration and a peak concentration (Cₘₐₓ) of meloxicam which is comparable to the Cₘₐₓ of a standard commercial oral formulation of meloxicam (e.g., Mobic® 7.5 mg). In some embodiments, the pharmaceutical formulation provides effective pain control in a subject starting at about 15 minutes to 2 hours after administration and lasting for up to 24 hours after administration. In some embodiments, the formulation is selected from one of a capsule, a tablet or granule powder. In some embodiments, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

### BRIEF DESCRIPTION OF THE FIGURES

The present invention will be further explained with reference to the attached drawings, where like structures are referred to by like numerals throughout the several views. The drawings shown are not necessarily to scale, with emphasis instead generally being placed upon illustrating the principles of the present invention. Further, some features may be exaggerated to show details of particular components.

As used herein, “mild to moderate acute pain” refers to a typically used terminology in pain management. Mild to moderate acute pain is a quantification of pain when an individual measures his pain as less than 7 on a scale of zero to 10. Mild to moderate acute pain can include back and neck pain, migraine, pain after surgery, etc. Mild to moderate acute pain also refers to pain lasting less than 3 months.

As used herein, “non-naïve” refers to an animal that has previously been used in earlier animal studies, e.g., but not limited to pharmacokinetics studies. As used herein, a “non-compartmental model” is a commonly used method analysis in pharmacokinetic studies. The model is highly dependent on estimation of total drug exposure. Total drug exposure is most often estimated by area-under-the-curve (AUC) methods, with the trapezoidal rule (numerical integration) the most common method.

The X-ray powder diffractogram (XRPD) studies in this invention were all conducted using a Bruker AXS D8 Advance Diffractometer. The experimental conditions: Cu-LKα radiation, voltage: 40 kV, current: 60 mA, 2θ range: 5-45°; diffractograms run: 4°/min. The diffractograms show a series of peaks collected at different scattering angles (scattering intensity vs. scattering angles at 2θ).

The diffraction scanning calorimetry (DSC) studies in this invention were all conducted using TA Q50. Temperature control was regulated in both ovens with a nitrogen flux at 50 ml/min; heating rate is 5° C./min from 25° C. to 300° C. The indium was used for instrument calibration. The amount of material used for analysis: 2 mg to 5 mg; the sample was placed in aluminum perforated pans.

The storage condition of 40° C./75% RH referred in this invention is a commonly used stress condition in pharmaceutical product development. The stress condition is intended to assess both physical and chemical stabilities of the test samples (e.g., “Intermediate”, formulation product, etc.). RH stands for “Relative Humidity”; “Open” refers to the test sample being freely exposed in such specified condition (e.g., 40° C./75% RH); “Closed” refers to the test sample being in an intended packaging or a container (e.g., HDPE bottle with aluminum seal) in such specified condition (e.g., 40° C./75% RH). The storage condition generally comes with a “time period” (e.g., 2 weeks or 2 months), indicating the actual time that the test sample has undergone in such specified storage condition. In our invention, the results of test samples from XRPD and DSC at both non-stressed condition and stressed conditions show that the meloxicam remain consistently stable amorphous in the meloxicam-cyclodextrin inclusion complex.

FIG. 1 is an XRPD showing the fingerprint of meloxicam.

FIG. 2 is an XRPD showing the fingerprint of an embodiment of a meloxicam-HPβCD inclusion complex (molar ratio: 1:2) of the present invention.

FIG. 3 is an XRPD showing the fingerprint of an embodiment of a meloxicam-HPβCD inclusion complex (molar ratio: 1:2) of the present invention. [storage condition: 40° C./75% RH, 2 weeks, open]

FIG. 4 is an XRPD showing the fingerprint of an embodiment of a meloxicam-HPβCD inclusion complex (molar ratio: 1:2) of the present invention. [storage condition: 40° C./75% RH, 2 months, closed]

FIG. 5 is an XRPD showing the fingerprint of an embodiment of a meloxicam-βCD inclusion complex (molar ratio: 1:2) of the present invention.

FIG. 6 is an XRPD showing the fingerprint of an embodiment of a meloxicam-βCD inclusion complex (molar ratio: 1:2) of the present invention. [storage condition: 40° C./75% RH, 2 weeks, open].

FIG. 7 is an XRPD showing the fingerprint of an embodiment of a meloxicam-βCD inclusion complex (molar ratio: 1:2) of the present invention. [storage condition: 40° C./75% RH, 2 months, closed]

FIG. 8 is a DSC curve showing the thermal transition of meloxicam.

FIG. 9 is a DSC curve showing the thermal transition of an embodiment of a meloxicam-HPβCD inclusion complex (molar ratio: 1:2) of the present invention.

FIG. 10 is a DSC curve showing the thermal transition of an embodiment of a meloxicam-HPβCD inclusion complex (molar ratio: 1:2) of the present invention. [storage condition: 40° C./75% RH, 2 weeks, open]

FIG. 11 is a DSC curve showing the thermal transition of an embodiment of a meloxicam-HPβCD inclusion complex (molar ratio: 1:2) of the present invention. [storage condition: 40° C./75% RH, 2 months, closed]

FIG. 12 is a DSC curve showing the thermal transition of an embodiment of a meloxicam-βCD inclusion complex (molar ratio: 1:2) of the present invention.

FIG. 13 is a DSC curve showing the thermal transition of an embodiment of a meloxicam-βCD inclusion complex (molar ratio: 1:2) of the present invention. [storage condition: 40° C./75% RH, 2 weeks, open]

FIG. 14 is a DSC curve showing the thermal transition of an embodiment of a meloxicam-βCD inclusion complex (molar ratio: 1:2) of the present invention. [storage condition: 40° C./75% RH, 2 months, closed]

FIG. 15 is a graph showing the comparison of plasma concentration over time after administering to non-naïve beagle dogs an embodiment of a meloxicam formulation of the present invention (Meloxicam-βCD Capsule 7.5 mg, Meloxicam-βCD Suspension 7.5 mg, Mobic® Tablet 7.5 mg). Additional description can be found in Example C1, provided herein.

FIG. 16 is a graph illustrating comparative pharmacokinetics profiles of some embodiments of meloxicam formulations of the present invention (Meloxicam-βCD Capsule 7.5 mg, Meloxicam-HPβCD Capsule 7.5 mg, Meloxicam-HPβCD Capsule 6 mg, Meloxicam-HPβCD Capsule 5 mg, and Mobic® Tablet 7.5 mg). Additional description can be found in Example C2, provided herein.

FIG. 17 is a graph illustrating comparative pharmacokinetics profiles and dose proportionality study of some embodiments of meloxicam formulations of the present invention which contain meloxicam-HPβCD inclusion complex (“Meloxicam-HPβCD Formulation”) after single oral administration to male and female non-naïve beagle dogs at both 5.5 mg (one capsule) and 11.0 mg (2 capsules) dose levels, and in comparison with meloxicam commercial product (Mobic® Tablet 7.5 mg). More details are presented in Example C3.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DESCRIPTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiments, though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

In an embodiment, the present invention is a method of administering a meloxicam formulation to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours (e.g., but not limited to, 0.25 hours, 0.5 hours, 1 hour, 1.5 hours, 2 hours, 2.5 hours, 3.0 hours). In an embodiment, the oral solid dosage form further includes one or more pharmaceutically acceptable excipients. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the oral solid dosage form is selected from one of a capsule, a tablet, a sachet, or granule powder. In an embodiment, the method of the present invention is for treating mild to moderate acute pain. In an embodiment, the mammalian subject is a human. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In an embodiment, the amorphous meloxicam-cyclodextrin inclusion complex displays no characteristic endothermic peak of crystalline meloxicam at 250° C.-258° C. as evidenced by differential scanning calorimetry.

In an embodiment, the present invention is a method of administering meloxicam to a mammalian subject to manage mild to moderate acute pain in the subject including: orally administering to the subject an oral pharmaceutical formulation including an amorphous meloxicam-cyclodextrin inclusion complex, where upon administration in the subject and after an amount of meloxicam has been released from the formulation, is absorbed by the subject, and reaches the systemic circulation of the subject, a shorter Tₘₐₓ is achieved as compared with a standard commercial formulation of meloxicam. In an embodiment, the shorter Tₘₐₓ is not greater than about 75% of the Tₘₐₓ exhibited by a standard commercial formulation of meloxicam. In an embodiment, the shorter Tₘₐₓ is not greater than about 50% of the Tₘₐₓ exhibited by a standard commercial formulation of meloxicam. In an embodiment, the shorter Tₘₐₓ is not greater than about 25% of the Tₘₐₓ exhibited by a standard commercial formulation of meloxicam. In an embodiment, upon administration in the subject and after an amount of meloxicam has been released from the formulation, is absorbed by the subject, and reaches the systemic circulation of the subject, a comparable Cₘₐₓ (e.g., 80-125%) is achieved as compared with that of a standard commercial formulation of meloxicam. In an embodiment, the formulation further includes one or more pharmaceutically acceptable excipients. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the formulation is selected from one of a capsule, a tablet, a sachet or granule powder. In an embodiment, the mammalian subject is a human. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In an embodiment, the amorphous meloxicam-cyclodextrin inclusion complex displays no characteristic endothermic peak of crystalline meloxicam at 250° C.-258° C. as evidenced by differential scanning calorimetry.

In an embodiment, the present invention is a solid pharmaceutical formulation including an effective amount of meloxicam, and one or more pharmaceutically acceptable excipients, the formulation including an amorphous meloxicam-cyclodextrin inclusion complex sufficiently designed to provide a time of meloxicam peak plasma concentration (Tₘₐₓ) of not greater than about 2 hours after administration and a peak concentration (Cₘₐₓ) of meloxicam which is higher than Cₘₐₓ of a standard commercial formulation of meloxicam. In an embodiment, the pharmaceutical formulation provides effective pain control in a subject starting at about 15 minutes to 2 hours after administration and lasting for up to 24 hours after administration. In an embodiment, the cyclodextrin is β-cyclodextrin. In an embodiment, the cyclodextrin is a derivative of β-cyclodextrin. In an embodiment, the cyclodextrin is hydroxylpropyl-β-cyclodextrin. In an embodiment, the formulation is selected from one of a capsule, a tablet, a sachet or granule powder. In an embodiment, the amorphous meloxicam-cyclodextrin inclusion complex displays typical amorphous halo and no characteristic peaks of crystalline meloxicam at 13.1, 14.9, 18.6, 25.9° at 2θ scales as evidenced by an Xray powder diffractogram. In an embodiment, the amorphous meloxicam-cyclodextrin inclusion complex displays no characteristic endothermic peak of crystalline meloxicam at 250° C.-258° C. as evidenced by differential scanning calorimetry.

In an embodiment, the present invention is directed to a method of orally administering a meloxicam formulation to a mammalian subject for the treatment of mild to moderate acute pain and/or inflammation, with the formulation including a meloxicam-cyclodextrin inclusion complex. In an embodiment, after administration of the formulation, a fast onset of therapeutic effect in the subject is achieved. In an embodiment, the administration of the meloxicam formulation results in a shorter Tₘₐₓ as compared to Tₘₐₓ of a standard commercial oral formulation of meloxicam. In an embodiment, the therapeutic effect is to treat mild to moderate acute pain and/or inflammation. In an embodiment, the formulation produces a Tₘₐₓ not greater than about 75% of the Tₘₐₓ exhibited by a standard commercial oral formulation of meloxicam. In an embodiment, the formulation produces a Tₘₐₓ not greater than about 50% of the Tₘₐₓ exhibited by a standard commercial oral formulation of meloxicam. In an embodiment, the formulation produces a Tₘₐₓ not greater than about 25% of the Tₘₐₓ exhibited by a standard commercial oral formulation of meloxicam. In an embodiment, the strength of meloxicam in a formulation of the present invention is not greater than about 80% of the strength of meloxicam in a standard commercial oral formulation. In an embodiment, the strength of meloxicam in a formulation of the present invention is not greater than about 70% of the strength of meloxicam in a standard commercial oral formulation. In an embodiment, the strength of meloxicam in a formulation of the present invention is not greater than about 60% of the strength of meloxicam in a standard commercial oral formulation. In an embodiment, the meloxicam-cyclodextrin inclusion complex at a formulation of the present invention is prepared through a spray drying process. In an embodiment, meloxicam in the inclusion complex is in an amorphous state. In an embodiment, meloxicam in the inclusion complex displays typical amorphous halo and no characteristic peaks of crystalline meloxicam such as 13.1, 14.9, 18.6, 25.9° at 2θ scales, as evidenced by an X-ray powder diffractogram (XRPD). In an embodiment, meloxicam in the inclusion complex displays no characteristic endothermic peak of crystalline meloxicam at 250-258° C. as evidenced by differential scanning calorimetry (DSC). In an embodiment, the meloxicam formulation further includes pharmaceutically acceptable excipients. In an embodiment, the meloxicam formulation is in an oral dosage form of a capsule. In an embodiment, the meloxicam formulation is in an oral dosage form of a tablet. In an embodiment, the meloxicam formulation is in an oral dosage form of granule powder.

According to aspects illustrated herein, there is disclosed a method to administer a meloxicam formulation to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours (e.g., but not limited to, 0.25 hours, 0.5 hours, 1 hour, 1.5 hours, 2 hours, 2.5 hours, 3.0 hours). In some embodiments, the oral solid dosage form further includes one or more pharmaceutically acceptable excipients. In some embodiments, the cyclodextrin is β-cyclodextrin. In some embodiments, the cyclodextrin is a derivative of β-cyclodextrin. In an embodiment, the cyclodextrin is hydroxylpropyl-β-cyclodextrin. In some embodiments, the oral solid dosage form is a capsule. In some embodiments, the oral solid dosage form is a tablet. In some embodiments, the oral solid dosage form is granule powder. In some embodiments, the meloxicam formulation is used to treat mild to moderate acute pain and/or inflammation. In some embodiments, the mammalian subject is a human. In some embodiments, the amorphous meloxicam-cyclodextrin inclusion complex displays a typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

In an embodiment, the present invention is directed to a method to administer meloxicam to a mammalian subject to manage mild to moderate acute pain, and/or inflammation in the subject including: orally administering to the subject an oral pharmaceutical formulation including an amorphous meloxicam-cyclodextrin inclusion complex, where upon administration in the subject and after an amount of meloxicam has been released from the formulation, is absorbed by the subject, and reaches the systemic circulation of the subject, a shorter Tₘₐₓ is achieved as compared with a standard commercial oral formulation of meloxicam. In an embodiment, the shorter Tₘₐₓ is not greater than about 75% of the Tₘₐₓ exhibited by a standard commercial formulation of meloxicam. In some embodiments, the shorter Tₘₐₓ is not greater than about 50% of the Tₘₐₓ exhibited by a standard commercial oral formulation of meloxicam. In some embodiments, the shorter Tₘₐₓ is not greater than about 25% of the Tₘₐₓ exhibited by a standard commercial oral formulation of meloxicam. In some embodiments, upon administration in the subject and after an amount of meloxicam has been released from the formulation, meloxicam released from the formulation is absorbed by the subject, and reaches the systemic circulation of the subject, a higher Cₘₐₓ (peak plasma concentration) is achieved as compared with a standard commercial oral formulation of meloxicam. In some embodiments, the formulation further includes one or more pharmaceutically acceptable excipients. In some embodiments, the cyclodextrin is β-cyclodextrin. In some embodiments, the cyclodextrin is a derivative of β-cyclodextrin. In an embodiment, the cyclodextrin is hydroxylpropyl-β-cyclodextrin. In some embodiments, the formulation is selected from one of a capsule, a tablet or granule powder. In some embodiments, the mammalian subject is a human. In some embodiments, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

In some embodiments, the present invention is directed to a solid pharmaceutical formulation including an effective amount of meloxicam, and one or more pharmaceutically acceptable excipients, the formulation including an amorphous meloxicam-cyclodextrin inclusion complex sufficiently designed to provide a time of meloxicam peak plasma concentration (Tₘₐₓ) of not greater than about 3 hours after administration and a peak concentration (Cₘₐₓ) of meloxicam which is comparable to the Cₘₐₓ of a standard commercial oral formulation of meloxicam. In some embodiments, the pharmaceutical formulation provides effective pain control in a subject starting at about 15 minutes to 2 hours after administration and lasting for up to 24 hours after administration. In some embodiments, the formulation is selected from one of a capsule, a tablet or granule powder. In some embodiments, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram. In some embodiments, the amorphous meloxicam-cyclodextrin inclusion complex displays no characteristic endothermic peak of crystalline meloxicam at 250° C.-258° C. as evidenced by differential scanning calorimetry.

As used herein, the term “crystalline” refers to the structural characteristics of meloxicam or the meloxicam in the meloxicam-cyclodextrin inclusion complex where there is a regular and/or repeating pattern in the structure, or in other words, there is long-range order. The term “amorphous” refers to the structural characteristics of meloxicam or the meloxicam in the meloxicam-cyclodextrin inclusion complex where there is an absence of regular and repeating pattern in the structure, or in other words, there is an absence of long-range order.

As used herein, the term “cyclodextrins” or “CD” refers to a cyclic compound including different number of alpha-(1-4) linked D-glucopyranosyl units: with 6 units being α-cyclodextrin (α-CD), 7 units being β-cyclodextrin (βCD), and 8 units being γ-cyclodextrin (γCD).

There are a variety of derivatives of βCD including hydroxypropyl-β-cyclodextrin (HPβCD) and sulfobutyl ether-β-cyclodextrin (SBEβCD). The HPβCD is a partially substituted poly(hydroxypropyl)ether-β-cycodextrin. The number of hydroxypropyl groups per anhydroglucose unit expressed as molar substitution is not less than 0.40 and not more than 1.50. SBEβCD is another commonly used βCD derivative, and is prepared by alkylation of βCD using 1,4-butane sultone under basic conditions. The average degree of substitution in βCD is not less than 6.2 and not more than 6.9.

As used herein, the term “meloxicam” refers to a compound with the chemical name 4-hydroxy-2-methyl-N-(5-methyl-2-thiazolyl)-2H-1,2-benzothiazine-3-carboxamide-1,1-dioxide and can be expressed as the structural formula:

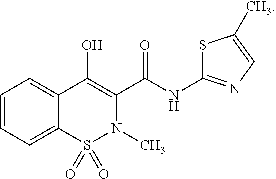 [SMILES: Cc1cnc(NC(=O)C2=C(O)c3ccccc3S(=O)(=O)N2C)s1]

As used herein, a “non-steroidal anti-inflammatory drug” or “NSAID” refers to a class of drugs that provides analgesic (pain-killing) and antipyretic (fever-reducing) effects, and, in higher doses, anti-inflammatory effects. An example of an NSAID is meloxicam, which can be delivered orally to a subject.

As used herein, the terms “oral meloxicam composition” or “meloxicam formulation” refer to oral dosage forms of the present invention including meloxicam. The oral dosage forms can include liquids (solutions, suspensions, and emulsions), semi-solids (pastes), and solids (tablets, capsules, powders, granules, premixes, and medicated blocks). In an embodiment, an oral meloxicam composition of the present invention is a capsule.

As used herein, the term “standard commercial oral formulation of meloxicam” refers to Mobic®, in the dosage form of Capsule or Tablet, or others, in the strength of 7.5 mg or 15 mg. “Standard commercial oral formulation of meloxicam” also refers to those bioequivalent or “generic” product of meloxicam, in the dosage form of Capsule or Tablet or others, and in the strength of 7.5 mg or 15 mg.

As used herein, “spray-drying” refers to a process involving breaking up liquid mixture into small droplets (atomization) and rapidly removing solvent from the mixture in a spray-drying chamber (or apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets.

As used herein, “inclusion complex” refers to a complex in which a drug molecule or a part of the drug molecule (“guest”) enters into the cavity of a cyclodextrin molecule (“host”).

As used herein, “meloxicam-cyclodextrin inclusion complex” refers to an embodiment that includes an inclusion complex formed between meloxicam and cyclodextrin (βCD or a derivative of the βCD) through a spray-drying process. If the complexation goes in full extent (meaning that all meloxicam molecules are complexed), meloxicam ceases to exist as crystalline, and will show amorphous characteristics in such an inclusion complex. Otherwise, it will show partially crystalline or full crystalline properties. In an embodiment, a variety of instrumentation techniques including ¹H-Nuclear Magnetic Resonance (¹H-NMR), ¹³C-Nuclear Magnetic Resonance (¹³C-NMR), Scanning Electronic Microscopy (SEM) and/or X-ray Powder Diffraction (XRRD), can be used to assess the effectiveness and stability of the inclusion complex formed between meloxicam and the cyclodextrin.

As used herein, “Spray Drying Intermediate” (or “Intermediate”) refers to an embodiment that includes the “meloxicam-cyclodextrin inclusion complex”. The “Intermediate” may also include certain excipient(s) or chemical(s) used in the spray-drying process. These excipients or chemicals are intended to promote the formation and stability of the complexation process and/or the complex that is formed. In this invention, “Spray Drying Intermediate” (or “Intermediate”) and “meloxicam-cyclodextrin inclusion complex” are often interchangeably used. However, there are some differences: “Spray Drying Intermediate” (or “Intermediate”) is a term used when describing formulation composition, while “meloxicam-cyclodextrin inclusion complex” is a term used when describing the molecular nature of an embodiment.

As used herein, a “substance concentration” refers to a total weight of ingredients (solid) in a spray solution which may include meloxicam, a cyclodextrin, an alkalizer (e.g.: sodium phosphate), a surfactant, a polymer, or any combination thereof. In some embodiments, the substance concentration can be measured by weight over volume (w/v).

As used herein, “Tₘₐₓ” refers to the time after administration of a drug compound when the maximum plasma concentration is achieved. As used herein, “Cₘₐₓ” refers to the peak plasma concentration of a drug compound after administration. As used herein, “strength” refers to the drug amount in a defined unit of a dosage form such as a capsule or a tablet. As used herein, “Dose” refers to the amount of a drug compound administered. As used herein, “AUC” or “area under the curve” refers to the area under the plot of plasma concentration of drug against time after drug administration, or an integral of the concentration-time curve. As used herein, “Bioavailability” refers to the systematically available fraction of a drug compound.

In an embodiment, a method includes administering a meloxicam formulation disclosed herein to a mammalian subject in need thereof including: orally administering to the subject an oral solid dosage form including an amorphous meloxicam-cyclodextrin inclusion complex, where administering the amorphous meloxicam-cyclodextrin inclusion complex results in the subject achieving a Tₘₐₓ not greater than about 3.0 hours (e.g., but not limited to, 0.25 hours, 0.5 hours, 1 hour, 1.5 hours, 2 hours, 2.5 hours, 3.0 hours).

In some embodiments, the oral solid dosage form further includes one or more pharmaceutically acceptable excipients. The excipient(s) may include but not limited to the following: the filler(s), the binder(s), the disintegrant(s), the lubricant(s), the surfactant(s), the glidant(s), the anti-oxidant(s), and any combination of these excipients.

In some embodiments, the inventive compositions can include at least one pharmaceutical excipient. In some embodiments, the at least one pharmaceutical excipient includes at least one filler, where the at least one filler can be a microcrystalline cellulose (MCC), a binder, a disintegrant, a lubricant, a surfactant, a glidant, an anti-oxidant, or any combination thereof. In some embodiments, at least one pharmaceutical excipient includes lactose monohydrate, crospovidone, magnesium stearate, or any combination thereof.

In some embodiments, the cyclodextrin is β-cyclodextrin. In some embodiments, the cyclodextrin is a derivative of β-cyclodextrin. In some embodiments, the derivative of β-cyclodextrin is hydroxylpropyl-β-cyclodextrin (HPβCD). In some embodiments, the derivative of β-cyclodextrin is sulfobutylether-β-cyclodextrin (SBEβCD). In some embodiments, the derivative of β-cyclodextrin is methyl-β-cyclodextrin. In some embodiments, the derivative of β-cyclodextrin is mercapto-β-cyclodextrin. In some embodiments, the derivative of β-cyclodextrin is benzyl-β-cyclodextrin. In some embodiments, the derivative of β-cyclodextrin is oligo (lactic acid)-β-cyclodextrin.

In some embodiments, the oral solid dosage form is selected from one of a capsule, a tablet, or granule powder.

In some embodiments, the method can be used for treating mild to moderate acute pain, and/or inflammation. In some embodiments, the mammalian subject is a human.

In some embodiments, the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram.

In some embodiments, the amorphous meloxicam-cyclodextrin inclusion complex displays no characteristic endothermic peak of crystalline meloxicam at 250° C.-258° C. as evidenced by differential scanning calorimetry.

In an embodiment, a method includes administering a meloxicam formulation disclosed herein to a mammalian subject to manage mild to moderate acute pain, and/or inflammation in the subject including: orally administering to the subject an oral pharmaceutical formulation including an amorphous meloxicam-cyclodextrin inclusion complex, where upon administration in the subject and after an amount of meloxicam has been released from the formulation, is absorbed by the subject, and reaches the systemic circulation of the subject, a shorter Tₘₐₓ is achieved as compared with a standard commercial oral formulation of meloxicam at the same or decreased dosage strength. In some embodiments, the shorter Tₘₐₓ is not greater than about 75% of the Tₘₐₓ exhibited by a standard commercial oral formulation of meloxicam. In some embodiments, the shorter Tₘₐₓ is not greater than about 50% of the Tₘₐₓ exhibited by a standard commercial oral formulation of meloxicam. In some embodiments, the shorter Tₘₐₓ is not greater than about 25% of the Tₘₐₓ exhibited by a standard commercial oral formulation of meloxicam.

In some embodiments, upon administration in the subject and after an amount of meloxicam has been released from the formulation, meloxicam released from the formulation is absorbed by the subject, and reaches the systemic circulation of the subject, a higher Cₘₐₓ is achieved as compared with a standard commercial oral formulation of meloxicam.

In some embodiments, a formulation disclosed herein further includes one or more pharmaceutically acceptable excipients. The excipient(s) in the formulation may include but not limited to the following: the filler(s) (“filler” is also known as “diluent”), the binder(s), the disintegrant(s), the lubricant(s), the surfactant(s), the glidant(s), the anti-oxidant(s), or any combination of excipients from these excipient categories.

Suitable filler(s) may include but not limited to the following: lactose monohydrate, anhydrous lactose, sucrose, dextrose, mannitol, sorbitol, starch, cellulose (particularly microcrystalline cellulose), dihydro- or anhydro-calcium phosphate, calcium carbonate, calcium sulfate, etc.

Suitable binder(s) may include but not limited to the following: acacia, cellulose derivatives (e.g.: methylcellulose, carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, sorbitol, starch, pregelatinized starch, tragacanth, xanthane resin, alginates, magnesium-aluminum silicate, polyethylene glycol, bentonite, etc.

Suitable disintegrant(s) may include but not limited to the following: starch, pregelatinized starch, hydroxypropyl starch, sodium starch glycolate, sodium carboxymethylcellulose, croscarmellose sodium, microcrystalline cellulose, alginates, resins, surfactants, effervescent compositions, aqueous aluminum silicates, cross-linked polyvinylpyrrolidone, etc.

Suitable lubricant(s) may include but not limited to the following magnesium stearate, calcium stearate, talc, polyethylene glycol, polymers of ethylene oxide, sodium lauryl sulfate, magnesium lauryl sulfate, sodium oleate, sodium stearyl fumarate, DL-leucine, colloidal silicon dioxide, etc.

In some embodiments, an anti-oxidant is added to the formulation composition in order to increase the chemical stability of meloxicam in the formulation. In some embodiments, a meloxicam formulation of the present invention includes at least one antioxidant, where the weight percent of the at least one antioxidant ranges from 0.05% to 2.0% (w/w). In an embodiment, the at least one antioxidant is selected from the group consisting of ascorbic acid, ascorbyl palmitate, butylated hydroxyanisole, butylated hydroxytoluene, stannous chloride, erythorbic acid, hypophosphorous acid, monothioglycerol, potassium metabisulfite, propyl gallate, sodium bisulfite, sodium formaldehyde sulfoxylate, sodium metabisulfite, sodium sulfite, sodium thiosulfate, sulfur dioxide, erythorbic acid, hypophosphorous acid, lactobionic acid, monothioglycerol, potassium metabisulfite, propyl gallate, racemethionine, stannous chloride, tocopherol, or any combination thereof.

In some embodiments, the formulation is in the dosage form of capsules, tablets, or granule powder.

In some embodiments, the mammalian subject is a human.

In an embodiment, a composition of the present disclosure is a solid pharmaceutical formulation including an effective amount of meloxicam, and one or more pharmaceutically acceptable excipients, the formulation including an amorphous meloxicam-cyclodextrin inclusion complex sufficiently designed to provide a time of meloxicam peak plasma concentration (Tₘₐₓ) of not greater than about 3 hours after administration and a peak concentration (Cₘₐₓ) of meloxicam which is comparable to the Cₘₐₓ of a standard commercial oral formulation of meloxicam.

In some embodiments, the pharmaceutical formulation provides effective pain control in a subject starting at about 15 minutes to 3 hours after administration and lasting for up to 24 hours after administration.

In some embodiments, a composition of the present invention is an oral meloxicam formulation that is available in dosage strengths that are lower than or the same as the standard commercial oral formulation of meloxicam (such as Mobic® formulation). In an embodiment, an oral meloxicam formulation of the present invention has a dosage strength of 4 mg. In an embodiment, an oral meloxicam formulation of the present invention has a dosage strength of 4.5 mg. In an embodiment, an oral meloxicam formulation of the present invention has a dosage strength of 5 mg. In an embodiment, an oral meloxicam formulation of the present invention has a dosage strength of 5.5 mg. In an embodiment, an oral meloxicam formulation of the present invention has a dosage strength of 6 mg. In an embodiment, an oral meloxicam formulation of the present invention has a dosage strength of 6.5 mg. In an embodiment, an oral meloxicam formulation of the present invention has a dosage strength of 7.0 mg. In an embodiment, an oral meloxicam formulation of the present invention has a dosage strength of 7.5 mg. In an embodiment, an oral meloxicam formulation of the present invention has a dosage strength of 8 mg. In an embodiment, an oral meloxicam formulation of the present invention has a dosage strength of 9 mg. In an embodiment, an oral meloxicam formulation of the present invention has a dosage strength of 10 mg. In an embodiment, an oral meloxicam formulation of the present invention has a dosage strength of 11 mg. In an embodiment, an oral meloxicam formulation of the present invention has a dosage strength of 12 mg. In an embodiment, an oral meloxicam formulation of the present invention has a dosage strength of 13 mg. In an embodiment, an oral meloxicam formulation of the present invention has a dosage strength of 14 mg. In an embodiment, an oral meloxicam formulation of the present invention has a dosage strength of 15 mg.

In some embodiments, when these lower or equivalent dosage strength oral meloxicam formulations of the present invention are administered to a subject, and absorbed into the general circulation, a reduced Tₘₐₓ is observed as compared to a Mobic® formulation having the same dosage strength or a higher dosage strength. In some embodiments, when a lower dosage strength oral meloxicam formulation of the present invention is administered to a subject, and absorbed into the circulation, a higher or a comparable Cₘₐₓ is observed as compared to a Mobic® formulation having the same dosage strength or a higher dosage strength. As used herein, the term “comparable” refers to a Cₘₐₓ and/or AUC in the range of 80%-125% as compared to those of Mobic® formulation having the same dosage strength or higher dosage strength. In some embodiments, a higher Cₘₐₓ and/or higher AUC means that the Cₘₐₓ and/or AUC is from 101%-125% as compared to a Mobic® formulation having the same dosage strength or a higher dosage strength. In some embodiments, when a lower strength oral meloxicam formulation of the present invention is administered to a subject, and absorbed into the circulation, a higher or a comparable AUC is observed as compared to a Mobic® formulation having the same dosage strength or a higher dosage strength. In some embodiments, a lower Cₘₐₓ and/or lower AUC means that the Cₘₐₓ and/or AUC is from 80%-99% as compared to a Mobic® formulation having the same dosage strength or a lower dosage strength. In some embodiments, when a lower strength (e.g.: 5.5 mg) formulation of the present invention is administered, a lower Tₘₐₓ is achieved, but exhibits a comparable Cₘₐₓ as compared with the standard oral commercial formulation of meloxicam (e.g.: Mobic® 7.5 mg). In some embodiments, the inventive compositions include the inclusion complex of meloxicam-βCD. In some embodiments, the inventive compositions include the inclusion complex of meloxicam-HPβCD. In some embodiments, the inventive compositions include the inclusion complex of meloxicam-SBEβCD. In some embodiments, the meloxicam-cyclodextrin inclusion complex is formed using a spray drying process disclosed herein and the meloxicam remains in an amorphous state in such complex.

In an embodiment, a composition of the present disclosure having the amorphous meloxicam-cyclodextrin inclusion complex exhibits an improved dissolution in vitro as compared to formulations including crystalline meloxicam, as determined by in vitro dissolution data. In an embodiment, a composition of the present disclosure having the amorphous meloxicam complex exhibits an increased rate of absorption in vivo, as determined by in vivo pharmacokinetic data. In some embodiments, a composition of the present disclosure having the amorphous meloxicam complex is administered to a subject to treat mild and/or moderate acute pain and/or inflammation.

In some embodiments, the inventive compositions, when administered to a subject, and absorbed into the circulation, result in a reduced Tₘₐₓ as compared to standard commercial oral formulation of meloxicam (such as Mobic®) having equal or higher dosage strengths. In some embodiments, the reduced Tₘₐₓ of an oral meloxicam formulation of the present invention can be used to treat a subject with mild to moderate acute pain and/or inflammation because the time required to reach maximum concentration of meloxicam in the circulation is shorter than prior art meloxicam formulations.

In some embodiments, the inventive compositions, when administered to a subject, and absorbed into the circulation, result in an increased or comparable Cₘₐₓ as compared to standard commercial oral formulation of meloxicam having equal dosage strengths or higher dosage strengths.

In some embodiments, the inventive compositions, when administered to a subject, and absorbed into the circulation, result in an increased or comparable AUC as compared to standard commercial oral formulation of meloxicam having equal dosage strengths or higher dosage strengths.

Meloxicam is the active ingredient in pharmaceutical products currently marketed using the trademark Mobic® and in generic pharmaceutical products which is available as an oral tablet or a capsule in 7.5 mg and 15 mg strengths.

In some embodiments, the inventive compositions, when administered to a subject, and absorbed into the circulation, result in a reduction in conventional treatment-related adverse events, e.g., cardiovascular risk and/or gastrointestinal risks, e.g., upper gastrointestinal bleeding/ulcer, constipation, stomach cramping, indigestion, diarrhea, abdominal bloating (e.g., due to gas) nausea/vomiting, etc.

In some embodiments, the inventive compositions can be configured to deliver an immediate release of meloxicam to a subject. In some embodiments, the inventive compositions are administered to a subject once daily. In some embodiments, the phase identification of meloxicam suitable for use in an inventive composition can be evaluated using X-ray powder diffraction and/or differential scanning calorimetry.

In some embodiments, the inventive compositions include an amorphous inclusion complex (also referred to as “Spray-Dried Intermediate” or “Intermediate”), prepared using a spray drying process. In some embodiments, an aqueous solution including an alkalizer is used in the spray drying process, where the alkalizer(s) can be selected from the following: ammonium hydroxide, ethylamine, triethylamine, ethanediamine, tromethamine, lysine, arginine, histidine, sodium hydroxide, sodium phosphate, sodium acetate, sodium carbonate, sodium bicarbonate, meglumine, or any combination thereof. In some embodiments, a mixed solution containing (1) an aqueous solvent and (2) an organic solvent, where the organic solvent can be acetone and/or ethanol, is used in the spray drying process. In some embodiments, the solution can be spray-dried, where a powdered inclusion complex is obtained from this process. In some embodiments, the inventive compositions are generated by blending the Intermediate with at least one pharmaceutical excipient, exposing the Intermediate to dry granulation, where the exposure results in improved powder flow properties, and encapsulating and/or tableting, and/or packaging in an administrable dosage form.

Inclusion-Complex Preparation

In some embodiments, an amorphous inclusion complex (“spray-dried intermediate” or “Intermediate”) can be generated by a spray-drying process, where the spray-drying process can be configured to generate an amorphous inclusion complex of meloxicam with cyclodextrin, and where the amorphous inclusion complex of meloxicam can be in a stable amorphous state. In some embodiments, the spray-drying process involves the following steps: (a) dissolving meloxicam and a cyclodextrin, where the cyclodextrin can be βCD, a derivative of βCD such as HPβCD or SBEβCD, in a solution, where the solution can be an aqueous solution or in a mixed solution, where the mixed solution includes an aqueous solution and/or a solvent solution, where the pH of the solution can be alkaline, where the alkalizer can be selected from the following: ammonia, sodium phosphate, sodium hydroxide, meglumine, and where the solution can be heated and/or stirred, (b) delivering the solution to a drying chamber of a spray-dryer, where the drying chamber produces an Intermediate and (c) blending and/or granulating the Intermediate, where the blending and/or granulating includes pharmaceutically acceptable excipients, and generating oral solid dosage forms (e.g., encapsulating or tableting).

In some embodiments, the cyclodextrin is β-cyclodextrin. In some embodiments, the cyclodextrin is a derivative of β-cyclodextrin. In some embodiments, the derivative is hydroxylpropyl-β-cyclodextrin (HPβCD), or sulfobutylether-β-cyclodextrin (SBEβCD), or methyl-β-cyclodextrin, or mercapto-β-cyclodextrin, or benzyl-β-cyclodextrin, or oligo (lactic acid)-β-cyclodextrin.

In some embodiments, formulations including of meloxicam-HPβCD inclusion complex has an improved dissolution profile as compared with the formulations including meloxicam-βCD inclusion complex across a broad range of dissolution media including pH 1, pH 2, pH 4.5, pH 6.1, pH 6.8, pH 7.4.

In some embodiments, the inventive compositions include a molar ratio of meloxicam and cyclodextrin (illustrated as meloxicam:cyclodextrin), where the molar ratio is 1:1. In some embodiments, the inventive compositions include a molar ratio of meloxicam:cyclodextrin, where the molar ratio is 1:1.5. In some embodiments, the inventive compositions include a molar ratio of meloxicam:cyclodextrin, where the molar ratio is 1:2. In some embodiments, the inventive compositions include a molar ratio of meloxicam:cyclodextrin, where the molar ratio is 1:2.5. In some embodiments, the inventive compositions include a molar ratio of meloxicam:cyclodextrin, where the molar ratio is 1:3.

In some embodiments, the inventive compositions include a molar ratio of meloxicam:cyclodextrin ranging from 1:1.5 to 1:2.5. In some embodiments, the inventive compositions include a molar ratio of meloxicam:cyclodextrin ranging from 1:1 to 1:3.

In some embodiments, the spray solution includes a substance concentration, where the substance concentration is the combined weight of meloxicam, cyclodextrin, and solid alkaline agent (e.g.: sodium phosphate). In some embodiments, the substance concentration ranges from 5% to 30% (w/v). In some embodiments, the substance concentration ranges from 10% to 25% (w/v). In some embodiments, the substance concentration ranges from 5% to 30% (w/v). In some embodiments, the substance concentration ranges from 5% to 25% (w/v). In some embodiments, the substance concentration ranges from 5% to 20% (w/v). In some embodiments, the substance concentration ranges from 5% to 15% (w/v). In some embodiments, the substance concentration ranges from 5% to 10% (w/v). In some embodiments, the substance concentration ranges from 10% to 30% (w/v). In some embodiments, the substance concentration ranges from 15% to 30% (w/v). In some embodiments, the substance concentration ranges from 20% to 30% (w/v). In some embodiments, the substance concentration ranges from 25% to 30% (w/v).

In some embodiments, the spray solution includes acetone ranging from 5% to 30% (v/v). In some embodiments, the spray solution includes acetone ranging from 5% to 25% (v/v). In some embodiments, the spray solution includes acetone ranging from 5% to 20% (v/v). In some embodiments, the spray solution includes acetone ranging from 5% to 15% (v/v). In some embodiments, the spray solution includes acetone ranging from 5% to 10% (v/v). In some embodiments, the spray solution includes acetone ranging from 10% to 30% (v/v). In some embodiments, the spray solution includes acetone ranging from 15% to 30% (v/v). In some embodiments, the spray solution includes acetone ranging from 20% to 30% (v/v). In some embodiments, the spray solution includes acetone ranging from 25% to 30% (v/v).

In some embodiments, the spray solution includes ethyl alcohol ranging from 5% to 30% (v/v). In some embodiments, the spray solution includes ethyl alcohol ranging from 5% to 25% (v/v). In some embodiments, the spray solution includes ethyl alcohol ranging from 5% to 20% (v/v). In some embodiments, the spray solution includes ethyl alcohol ranging from 5% to 15% (v/v). In some embodiments, the spray solution includes ethyl alcohol ranging from 5% to 10% (v/v). In some embodiments, the spray solution includes ethyl alcohol ranging from 10% to 30% (v/v). In some embodiments, the spray solution includes ethyl alcohol ranging from 15% to 30% (v/v). In some embodiments, the spray solution includes ethyl alcohol ranging from 20% to 30% (v/v). In some embodiments, the spray solution includes ethyl alcohol ranging from 25% to 30% (v/v).

In some embodiments, the spray solution has a pH ranging from 8.0 to 11.0. In some embodiments, the spray solution has a pH ranging from 8.0 to 10.5. In some embodiments, the spray solution has a pH ranging from 8.0 to 10.0. In some embodiments, the spray solution has a pH ranging from 8.0 to 9.5. In some embodiments, the spray solution has a pH ranging from 8.0 to 9. In some embodiments, the spray solution has a pH ranging from 9.0 to 11.0. In some embodiments, the spray solution has a pH ranging from 9.5 to 11. In some embodiments, the spray solution has a pH ranging from 10.0 to 11. In some embodiments, the spray solution has a pH ranging from 8.0 to 10.0. In some embodiments, the spray solution has a pH ranging from 8.5 to 9.5.

While preparing spray solution, an alkalizer can be added, where the alkalizer is in the form of liquid or solid, or a combination of both, so as to adjust the solution pH to alkaline range to promote solubization of the drug compound (meloxicam). In some embodiments, the preparation of a spray solution includes an alkalizer, where the alkalizer is in the range of, e.g., but not limited to, 0.1-3.0% volume/volume (v/v) and/or weight/volume (w/v). In some embodiments, the alkalizer is in the range of 1.0-3.0% volume/volume (v/v) and/or weight/volume (w/v). In some embodiments, the alkalizer is in the range of 2.0-3.0% volume/volume (v/v) and/or weight/volume (w/v). In some embodiments, the alkalizer is in the range of 0.1-2.0% volume/volume (v/v) and/or weight/volume (w/v). In some embodiments, the alkalizer is in the range of 0.1-1.0% volume/volume (v/v) and/or weight/volume (w/v). In some embodiments, the alkalizer is in the range of 1.0-2.0% volume/volume (v/v) and/or weight/volume (w/v). In some embodiments, the alkalizer includes a liquid, where the liquid includes ammonium hydroxide, ethylamine, triethylamine, ethanediamine, etc. In some embodiments, the alkalizer includes a solid, where the solid includes lysine, arginine, histidine, sodium hydroxide, sodium phosphate, sodium acetate, sodium carbonate, meglumine, or any combination thereof.

In some embodiments, the spray solution includes ammonia hydroxide ranging from 0.3% to 3.0% (v/v). In some embodiments, the spray solution includes ammonia hydroxide ranging from 0.3% to 2.5% (v/v). In some embodiments, the spray solution includes ammonia hydroxide ranging from 0.3% to 2.0% (v/v). In some embodiments, the spray solution includes ammonia hydroxide ranging from 0.3% to 1.5% (v/v). In some embodiments, the spray solution includes ammonia hydroxide ranging from 0.3% to 1.0% (v/v). In some embodiments, the spray solution includes ammonia hydroxide ranging from 0.3% to 0.5% (v/v). In some embodiments, the spray solution includes ammonia hydroxide ranging from 0.5% to 3.0% (v/v). In some embodiments, the spray solution includes ammonia hydroxide ranging from 1.0% to 3.0% (v/v). In some embodiments, the spray solution includes ammonia hydroxide ranging from 1.5% to 3.0% (v/v). In some embodiments, the spray solution includes ammonia hydroxide ranging from 2.0% to 3.0% (v/v). In some embodiments, the spray solution includes ammonia hydroxide ranging from 2.5% to 3.0% (v/v).

In some embodiments, the spray solution includes meglumine ranging from 0.5-3.0% (w/v). In some embodiments, the spray solution includes meglumine ranging from 0.5% to 2.5% (w/v). In some embodiments, the spray solution includes meglumine ranging from 0.5% to 2.0% (w/v). In some embodiments, the spray solution includes meglumine ranging from 0.5% to 1.5% (w/v). In some embodiments, the spray solution includes meglumine ranging from 1.0% to 3.0% (w/v). In some embodiments, the spray solution includes meglumine ranging from 1.5% to 3.0% (w/v). In some embodiments, the spray solution includes meglumine ranging from 2.0% to 3.0% (w/v). In some embodiments, the spray solution includes meglumine ranging from 1.0% to 2.0% (w/v).

In some embodiments, the spray solution includes sodium hydroxide ranging from 0.1% to 2.0% (w/v). In some embodiments, the spray solution includes sodium hydroxide ranging from 0.1% to 1.5% (w/v). In some embodiments, the spray solution includes sodium hydroxide ranging from 0.1% to 1.0% (w/v). In some embodiments, the spray solution includes sodium hydroxide ranging from 0.5% to 2.0% (w/v). In some embodiments, the spray solution includes sodium hydroxide ranging from 1.0% to 2.0% (w/v). In some embodiments, the spray solution includes sodium hydroxide ranging from 0.5% to 1.5% (w/v).

In some embodiments, the spray solution includes triethylamine ranging from 0.05% to 1.0% (v/v). In some embodiments, the spray solution includes triethylamine ranging from 0.05% to 0.5% (v/v). In some embodiments, the spray solution includes triethylamine ranging from 0.05% to 0.1% (v/v). In some embodiments, the spray solution includes triethylamine ranging from 0.05% to 1.0% (v/v). In some embodiments, the spray solution includes triethylamine ranging from 0.1% to 1.0% (v/v). In some embodiments, the spray solution includes triethylamine ranging from 0.5% to 1.0% (v/v).

In some embodiments, the spray solution includes ethylamine ranging from 0.05% to 1.0% (v/v). In some embodiments, the spray solution includes ethylamine ranging from 0.05% to 0.5% (v/v). In some embodiments, the spray solution includes ethylamine ranging from 0.05% to 0.1% (v/v). In some embodiments, the spray solution includes ethylamine ranging from 0.05% to 1.0% (v/v). In some embodiments, the spray solution includes ethylamine ranging from 0.1% to 1.0% (v/v). In some embodiments, the spray solution includes ethylamine ranging from 0.5% to 1.0% (v/v).

In some embodiments, the spray solution includes ethanediamine ranging from 0.05% to 1.0% (v/v). In some embodiments, the spray solution includes ethanediamine ranging from 0.05% to 0.5% (v/v). In some embodiments, the spray solution includes ethanediamine ranging from 0.05% to 0.1% (v/v). In some embodiments, the spray solution includes ethanediamine ranging from 0.05% to 1.0% (v/v). In some embodiments, the spray solution includes ethanediamine ranging from 0.1% to 1.0% (v/v). In some embodiments, the spray solution includes ethanediamine ranging from 0.5% to 1.0% (v/v).

In some embodiments, the spray solution includes sodium phosphate ranging from 0.5% to 3.0% (w/v). In some embodiments, the spray solution includes sodium phosphate ranging from 0.5% to 2.5% (w/v). In some embodiments, the spray solution includes sodium phosphate ranging from 0.5% to 2.0% (w/v). In some embodiments, the spray solution includes sodium phosphate ranging from 0.5% to 1.5% (w/v). In some embodiments, the spray solution includes sodium phosphate ranging from 0.5% to 1.0% (w/v). In some embodiments, the spray solution includes sodium phosphate ranging from 1.0% to 3.0% (w/v). In some embodiments, the spray solution includes sodium phosphate ranging from 1.5% to 3.0% (w/v). In some embodiments, the spray solution includes sodium phosphate ranging from 2.0% to 3.0% (w/v). In some embodiments, the spray solution includes sodium phosphate ranging from 2.5% to 3.0% (w/v).

While preparing spray solution, it may be beneficial to add small quantities of surfactant(s), and/or polymer(s), in order to improve efficacy or effectiveness or stability of the meloxicam-cyclodextrin inclusion complex. In some embodiments, the spray solution may include at least one surfactant, where the surfactant in the concentration of 0.1-3.0% (w/v), and where the surfactant includes quaternary ammonium compounds (e.g., benzalkonium chloride, benzethonium chloride and cetylpyridinium chloride), dioctyl sodium sulfosuccinate, polyoxyethylene alkylphenyl ethers, poloxamers (e.g., polyoxyethylene and polyoxypropylene block copolymers), polyoxyethylene fatty acid glycerides, polyoxyethylene alkyl ethers (e.g., polyoxyethylene cetostearyl ether), polyoxyethylene fatty acid esters (e.g., polyoxyethylene stearate), polyoxyethylene sorbitan esters (e.g., polysorbate 20 and polysorbate 80), propylene glycol fatty acid esters (e.g., propylene glycol laurate), sodium lauryl sulfate, or any combination thereof. In some embodiments, the spray drying process includes at least one polymer, where the at least one polymer in the concentration of 0.1-3.0% (w/v), and where at least one polymer includes polyvinylpyrrolidone (PVP), hydroxypropylmethylcellulose (HPMC), HPMC phthalate, ethylcellulose, hydroxyethylcellulose, sodium carboxymethylcellulose (carmellose sodium), calcium carboxymethylcellulose, dextranacacia, starches (e.g., sodium starch glycolate), block copolymers of ethylene oxide and/or propylene oxide (e.g., PluronicTM F-68 and F-108), polyvinyl alcohol and polyethylene glycol (PEG), or any combination thereof.

In some embodiments, the spray solution can be heated at a temperature ranging from 45° C. to 85° C. In some embodiments, the spray solution can be heated at a temperature ranging from 55° C. to 80° C. In some embodiments, the spray solution can be heated at a temperature ranging from 65° C. to 75° C.

In some embodiments, the inlet temperature of the spray-dryer ranges from 100° C. to 180° C. In some embodiments, the inlet temperature of the spray-dryer ranges from 100° C. to 160° C. In some embodiments, the inlet temperature of the spray-dryer ranges from 110° C. to 140° C. In some embodiments, the inlet temperature of the spray-dryer ranges from 110° C. to 130° C.

In some embodiments, the Spray-Dried Intermediate (“Intermediate”) is further dried using oven drying, or tray drying, or fluid bed drying, or other drying techniques. In some embodiments, the drying ranges from 50° C. to 90° C. for a duration of 1 hour to 24 hours. In some embodiments, the drying ranges from 60° C. to 80° C. for a duration of 1 hour to 12 hours.

In some embodiments, the inventive compositions exhibit improved dissolution rates of meloxicam as compared to Mobic® compositions of meloxicam across a broad range of pH solutions including pH 1, pH 2, pH 3, pH 4.5, pH 6.1, pH 6.8, pH 7.4, and pH 8.0

The meloxicam-cyclodextrin inclusion complexes of the present invention are substantially free of crystalline meloxicam. In some embodiments, the quantitative measurement of “substantially free” can be less than 5%, less than 1%, less than 0.1%, and less than 0.01%. In some embodiments, the quantitative measurement of “substantially free” ranges from 0.01% to 5%. In some embodiments, the quantitative measurement of “substantially free” ranges from 0.1% to 5%. In some embodiments, the quantitative measurement of “substantially free” ranges from 0.5% to 5%. In some embodiments, the quantitative measurement of “substantially free” ranges from 1% to 5%. In some embodiments, the quantitative measurement of “substantially free” ranges from 0.01% to 1%. In some embodiments, the quantitative measurement of “substantially free” ranges from 0.01% to 0.5%. In some embodiments, the quantitative measurement of “substantially free” ranges from 0.01% to 0.1%.

In some embodiments, differential scanning calorimetry (DSC) and/or X-ray powder diffraction (XRPD) can be used to assess the absence/presence of meloxicam crystals in the meloxicam-cyclodextrin inclusion complex. In some embodiments, the inventive composition do not show the typical meloxicam endothermic peak around 250-258° C. using DSC. In some embodiments, the inventive compositions do not show a characteristic peak of crystalline meloxicam, where the characteristic peaks include, but are not limited to, major peaks at 13.1, 14.9, 18.6, 25.9° at 2θ scale.

In some embodiments, the formulation is including a composition ratio of an Intermediate versus pharmaceutical excipient that ranges from 10%:90% to 90%:10%. In some embodiments, the composition ratio of Intermediate vs. pharmaceutical excipient ranges from 20%: 80% to 80%:20%. In some embodiments, the ratio of Intermediate vs. pharmaceutical excipient ranges from 30%: 70% to 70%:30%. In some embodiments, the ratio of Intermediate vs. pharmaceutical excipient ranges from 40%: 60% to 60%:40%. In some embodiments, the ratio of Intermediate vs. pharmaceutical excipient ranges from 50%: 50% to 50%:50%.

### EXAMPLES

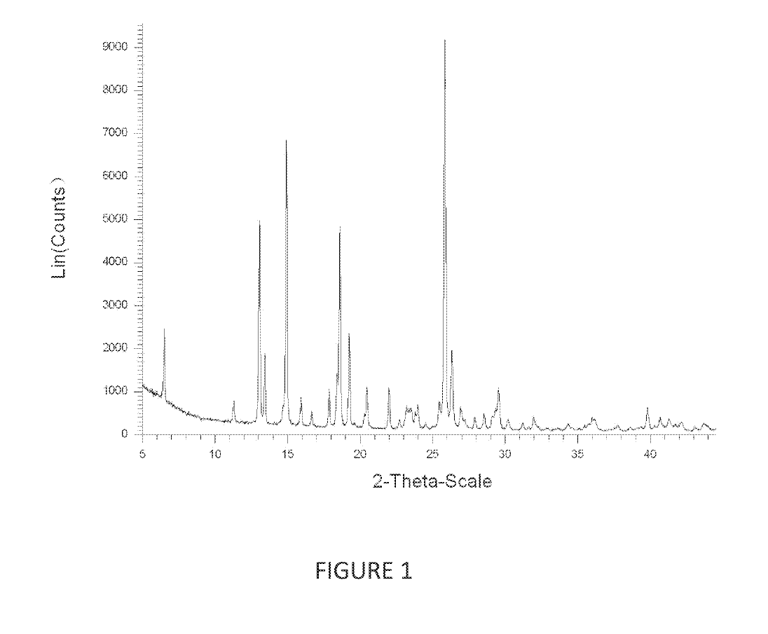

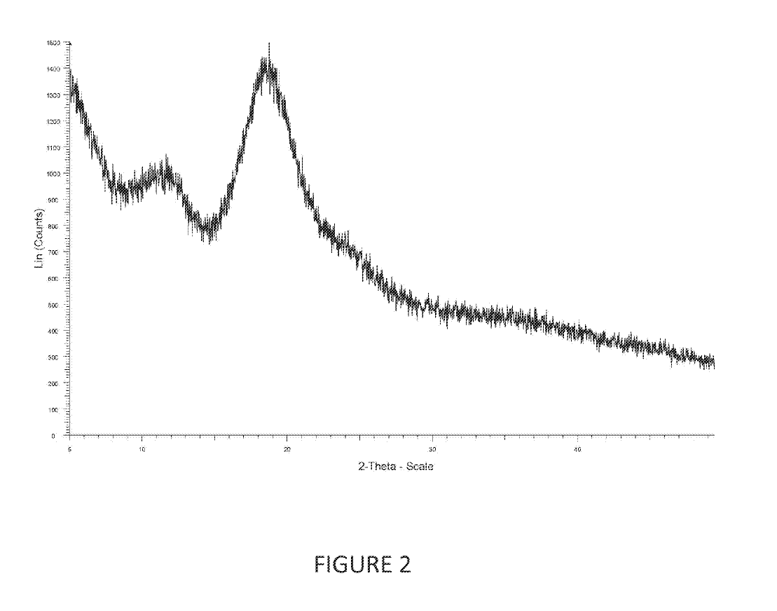

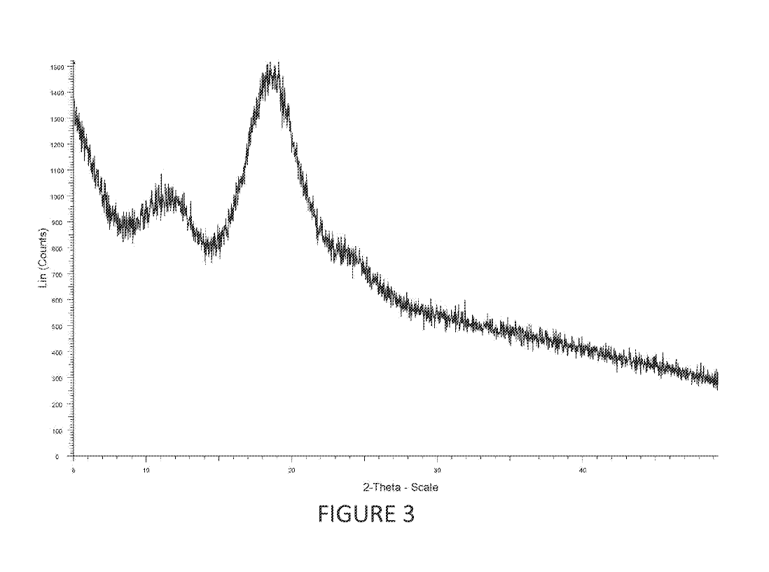

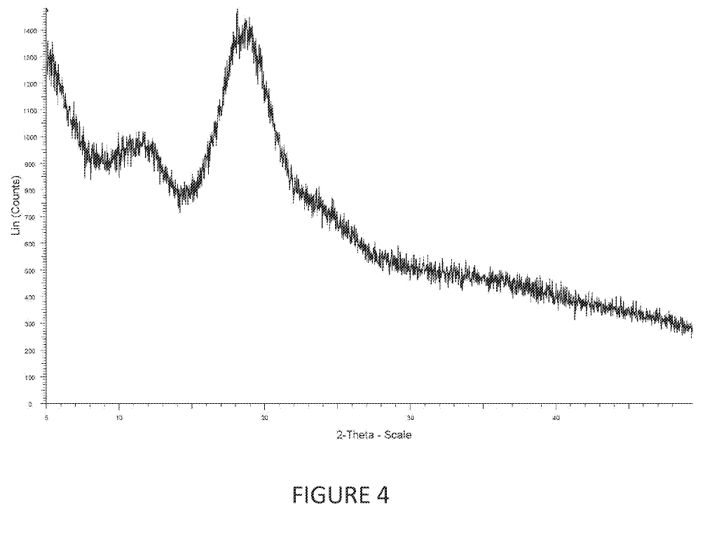

FIG. 1 is the XRPD showing the fingerprint of meloxicam as in crystalline state. FIG. 2 is the XRPD showing the meloxicam-HPβCD inclusion complex with a molar ratio of 1:2. FIG. 3 is the XRPD showing the same meloxicam-HPβCD inclusion complex (molar ratio: 1:2), as shown in FIG. 2, but undergone a specific stress condition [40° C./75% RH, 2 weeks, open]. FIG. 4 is the XRPD of the same meloxicam-HPβCD inclusion complex (molar ratio: 1:2), as shown in FIG. 2, but undergone a different stress condition [40° C./75% RH, 2 months, closed]. These figures confirm that, despite various and extensive stress conditions, meloxicam remains amorphous in the meloxicam-HPβCD inclusion complex. This indicates that the drug compound in the meloxicam-HPβCD inclusion complex remains stable amorphous, and that the spray drying preparation method disclosed in this invention is useful and effective in preparing such stable inclusion complex.

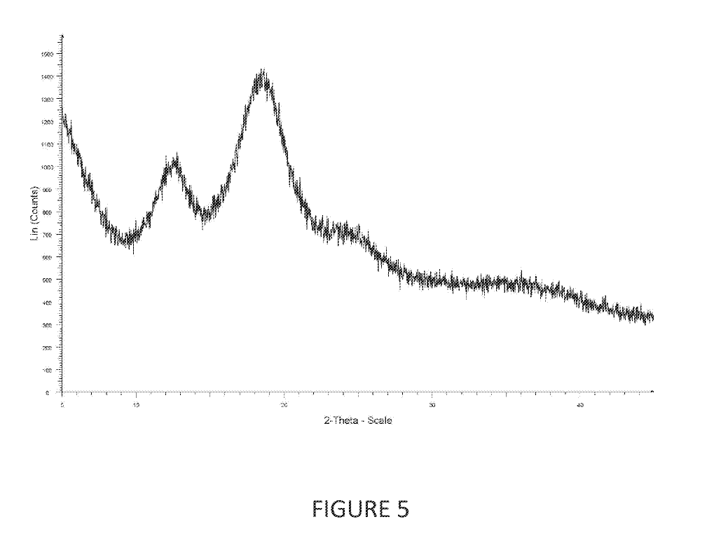

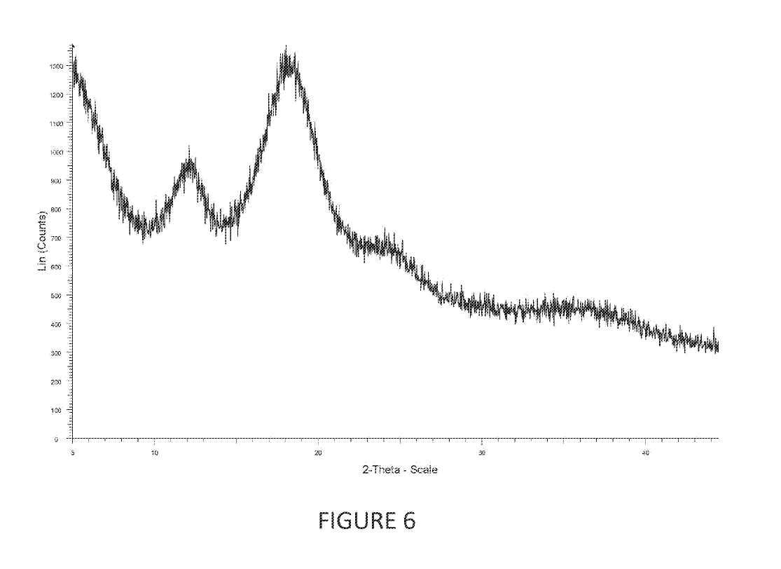

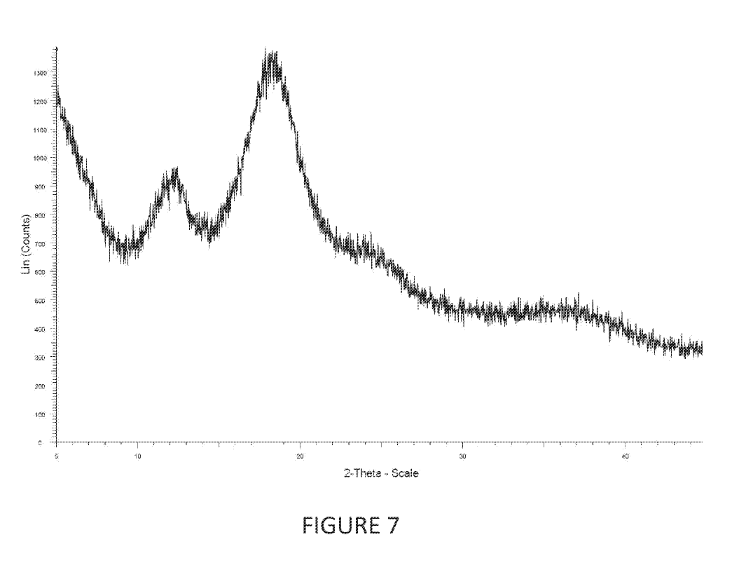

FIG. 5 is the XRPD showing meloxicam-βCD inclusion complex (molar ratio: 1:2). FIG. 6 is the XRPD showing the same meloxicam-βCD inclusion complex (molar ratio: 1:2), as shown in FIG. 5, but undergone a specific stress condition [40° C./75% RH, 2 weeks, open]. FIG. 7 is the XRPD of the same meloxicam-βCD inclusion complex (molar ratio: 1:2), as shown in FIG. 5, but undergone a different stress condition [40° C./75% RH, 2 months, closed]. These figures show that, despite various and extensive stress conditions, meloxicam remains amorphous in the meloxicam-βCD inclusion complex. This indicates that the drug compound in the meloxicam-βCD inclusion complex remains stable amorphous, and that the spray drying preparation method disclosed in this invention is useful and effective in preparing such stable inclusion complex.

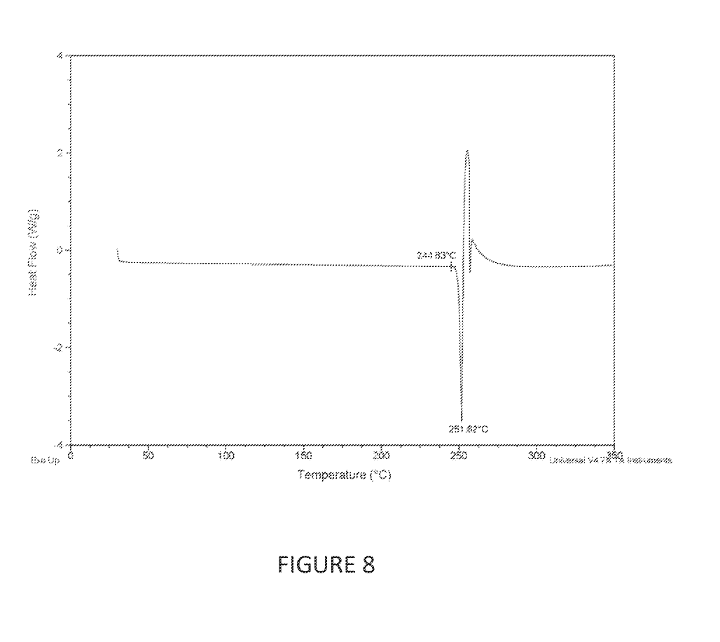

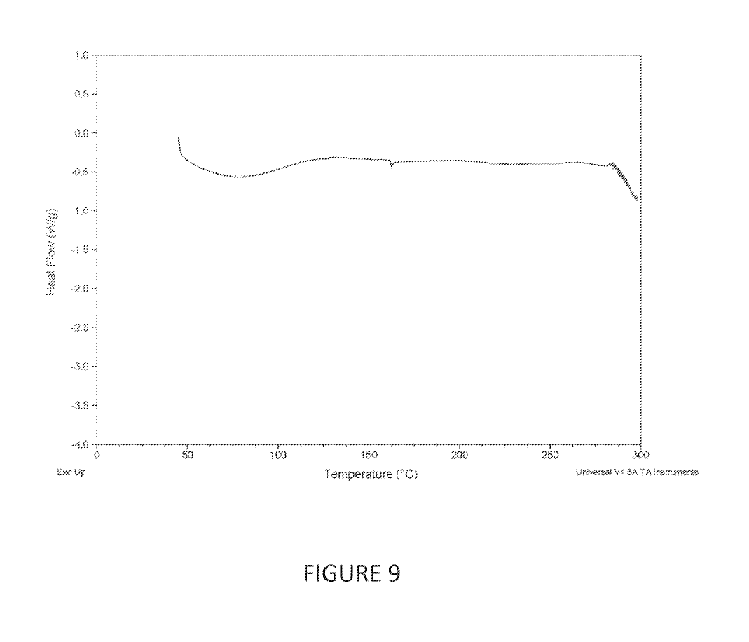

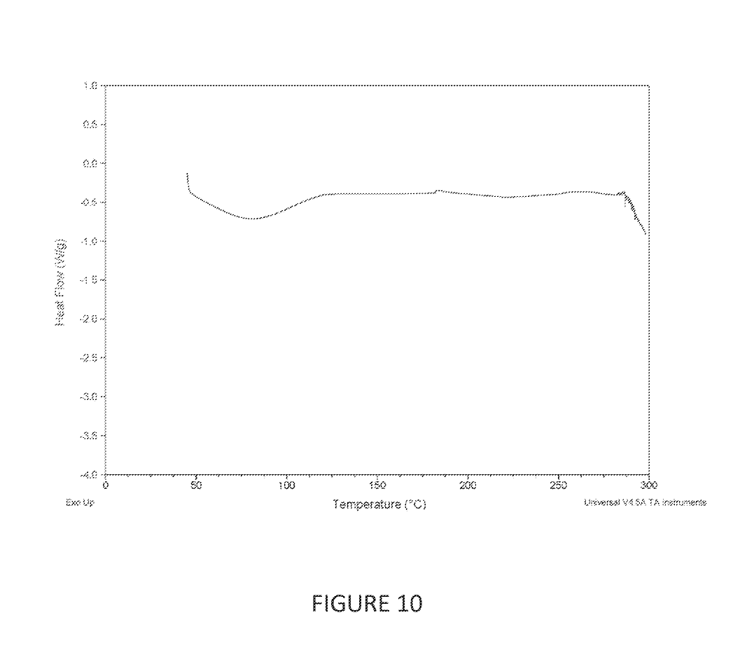

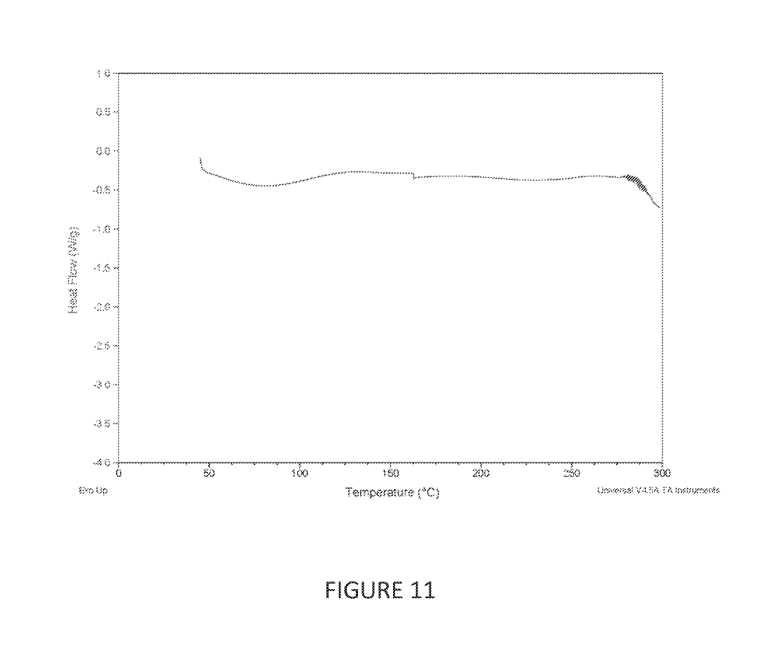

FIG. 8 is the DSC curve showing the thermal transition of meloxicam as in crystalline state. FIG. 9 is the DSC curve showing the meloxicam-HPβCD inclusion complex with a molar ratio of 1:2. FIG. 10 is the DSC curve showing the same meloxicam-HPβCD inclusion complex (molar ratio: 1:2), as shown in FIG. 9, but undergone a specific stress condition [40° C./75% RH, 2 weeks, open]. FIG. 11 is the DSC curve of the same meloxicam-HPβCD inclusion complex (molar ratio: 1:2), as shown in FIG. 9, but undergone a different stress condition [40° C./75% RH, 2 months, closed]. These figures show that, despite various and extensive stress conditions, meloxicam remains amorphous in the meloxicam-HPβCD inclusion complex. This indicates that the drug compound in the meloxicam-HPβCD inclusion complex remains stable amorphous, and that the spray drying preparation method disclosed in this invention is useful and effective in preparing such stable inclusion complex.

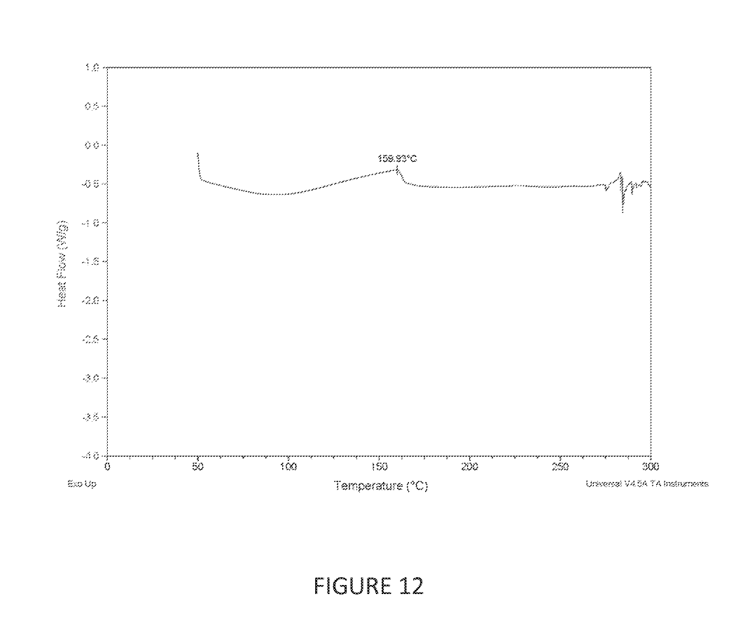

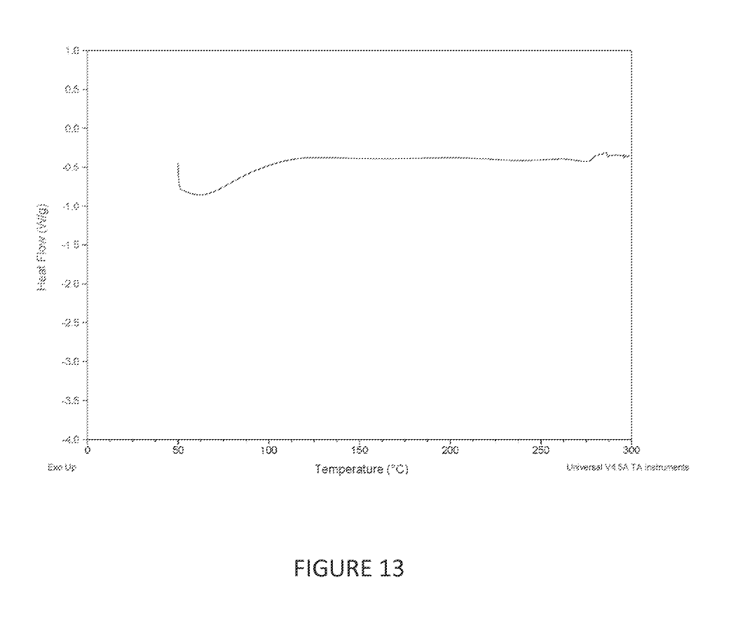

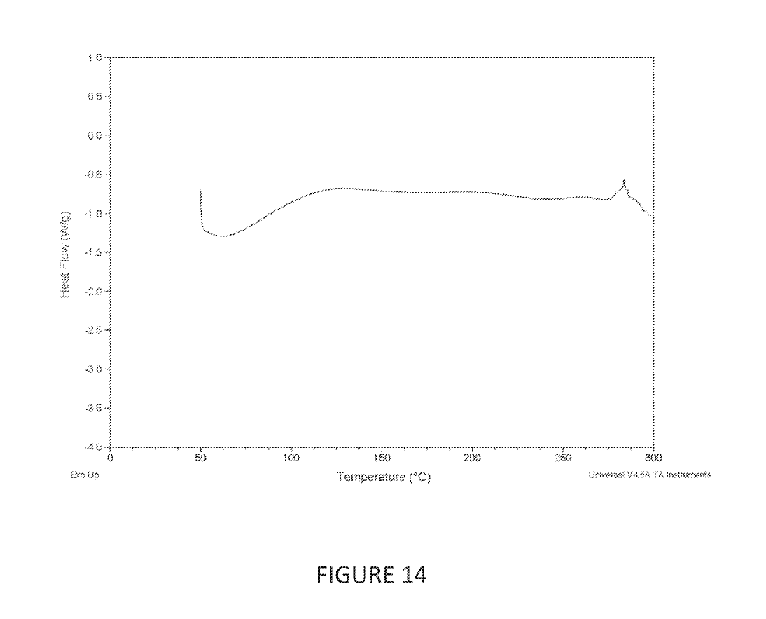

FIG. 12 is the DSC curve showing the meloxicam-βCD inclusion complex with a molar ratio of 1:2. FIG. 13 is the DSC curve showing the same meloxicam-βCD inclusion complex (molar ratio: 1:2), as shown in FIG. 12, but undergone a specific stress condition [40° C./75% RH, 2 weeks, open]. FIG. 14 is the DSC curve of the same meloxicam-HPβCD inclusion complex (molar ratio: 1:2), as shown in FIG. 12, but undergone a different stress condition [40° C./75% RH, 2 months, closed]. These figures show that, despite various and extensive stress conditions, meloxicam remains amorphous in the meloxicam-βCD inclusion complex. This indicates that the drug compound in the meloxicam-βCD inclusion complex remains stable amorphous, and that the spray drying preparation method disclosed in this invention is useful and effective in preparing such stable inclusion complex.

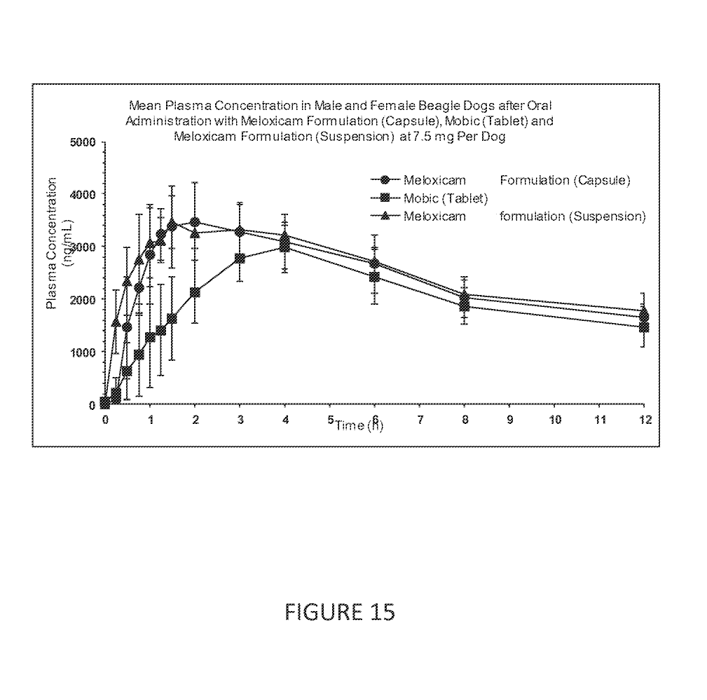

FIG. 15 is a graph showing the comparison of plasma concentration over time after administering to dogs embodiments of meloxicam formulations of the present invention ((Meloxicam-βCD Capsule 7.5 mg, Meloxicam-βCD Suspension 7.5 mg, Mobic® tablet 7.5 mg). After oral administration, plasma concentration was measured over a period of time (total: 48-hour; shown here only 12-hours in FIG. 15 for illustration purpose). Reduced Tₘₐₓ was observed in formulations disclosed in this invention as compared with Mobic®, all at 7.5 mg as administered to the dogs. Greater Cₘₐₓ was observed for formulations disclosed in this invention as compared with Mobic®, all at 7.5 mg as administered to the dogs (for example, e.g., see Example C1).

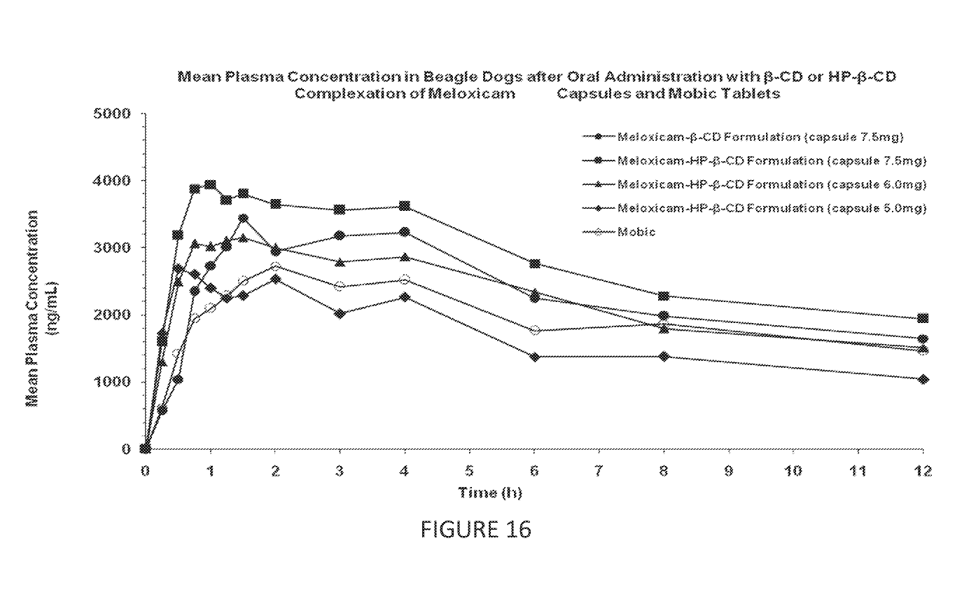

FIG. 16 is a graph showing the comparison of plasma concentration over time after administering to dogs embodiments of meloxicam formulations of the present invention (Meloxicam-βCD Capsule 7.5 mg, Meloxicam-HPβCD Capsule 7.5 mg, Meloxicam-HPβCD Capsule 6 mg, Meloxicam-HPβCD Capsule 5 mg, Mobic® Tablet 7.5 mg). After oral administration, the plasma concentration was measured over a period of time (total: 48-hour; shown here only 12-hours in FIG. 16 for illustration purpose). Reduced Tₘₐₓ was observed in all formulations (different cyclodextrins, and varying strengths) disclosed in this invention as compared with Mobic®. Greater Cₘₐₓ was observed for formulations disclosed in this invention as compared with Mobic®, all at the same strength as 7.5 mg (for example, e.g., see Example C2).

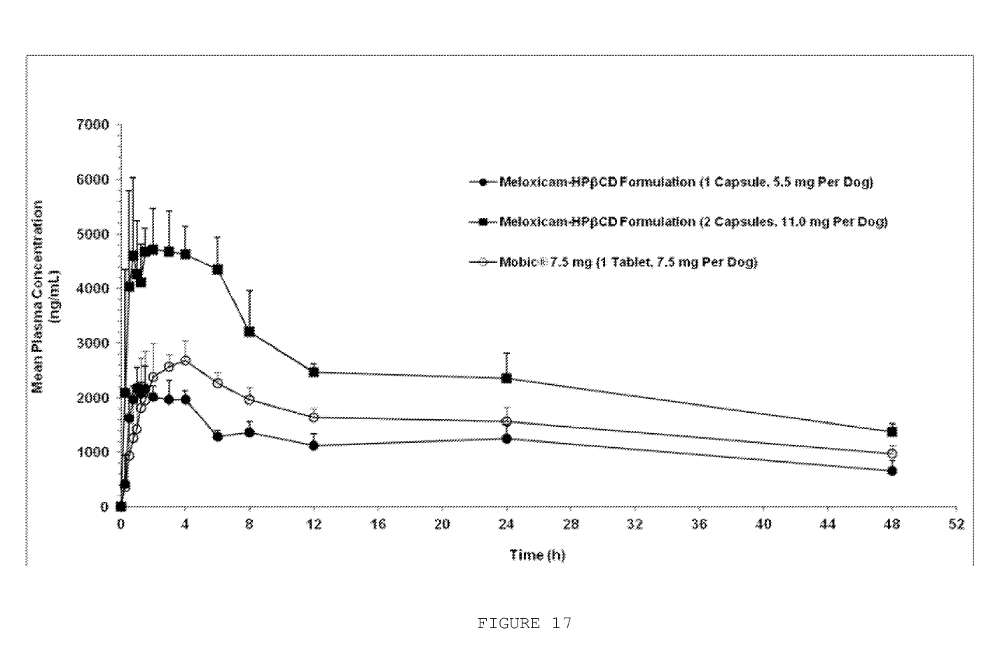

FIG. 17 is a graph showing the plasma concentration over time after administering to non-naïve beagle dogs with embodiments of Meloxicam-HPβCD Formulation of the present invention at doses of 5.5 mg (i.e., one capsule per dog) and 11.0 mg (2 capsules per dog). After oral administration, the plasma concentration was measured over a period of 48-hour. Reduced Tₘₐₓ was observed in Meloxicam-HPβCD Formulation as compared with Mobic®. Comparable exposure (Cₘₐₓ and AUC) was observed for Meloxicam-HPβCD formulation at 5.5 mg per dog, as compared with Mobic® tablet 7.5 mg. Further, as the dose increases from 5.5 mg to 11.0 mg per dog, the exposure (AUC and Cₘₐₓ) of the drug meloxicam increases proportionally (e.g., Example C3).

### Examples A1-A24

### Experimental Methods for Preparing a Spray Drying Intermediate (“Intermediate”)

The following examples provide methods and systems for preparing Spray Drying Intermediates (“Intermediate”) of the present invention. These examples illustrate a broad range of tested variables using the spray drying process; including, but not limited to, molar ratio of meloxicam versus cyclodextrin, varied cyclodextrin molecules (e.g., HPβCD, SBEβCD, βCD), spray solution preparation, and spray drying conditions.

### Example A1

### Molar Ratio (Meloxicam: HPβCD) of 1:1

72.0 g of HPβCD (Kleptose® HPB oral grade) was added to 300 ml water and heated to 70° C. 2 ml ammonia hydroxide solution (28-32%) was added to the solution. Then, 18 g meloxicam at a molar ratio (meloxicam: HPβCD) of 1:1 was added and fully dissolved in the solution. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 72.0 g; bulk density 0.28 g/ml; tap density: 0.30 g/ml; Angle of repose: 49°. State of amorphous/crystalline: XRPD: amorphous halo; DSC: absence of endothermic peak at 250-258° C.

### Example A2

### Molar Ratio (Meloxicam: HPβCD) of 1:1.5

107.0 g of HPβCD (Kleptose® HPB oral grade) was added to 400 ml water and heated to 70° C. 2 ml ammonia hydroxide solution (28-32%) was added to this solution. 18 g meloxicam at a molar ratio (meloxicam: HPβCD) of 1:1.5 was then added and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 100.0 g; bulk density 0.27 g/ml; tap density: 0.33 g/ml; Angle of repose: 53°. State of amorphous/crystalline: XRPD: amorphous halo; DSC: absence of endothermic peak at 250-258° C.

### Example A3

### Molar Ratio (Meloxicam: HPβCD) of 1:2

144.0 g of HPβCD (Kleptose® HPB oral grade) was added to 500 ml water and heated to 75° C. 3 ml ammonia hydroxide solution (28-32%) was then added to the solution. 18 g meloxicam at a molar ratio (meloxicam: HPβCD) of 1:2 was added to the solution and fully dissolved. Parameters used for spray drying process: Buchi Mini Spray Dryer B-290; inlet temp: 125° C.; outlet temp: 72-75° C.; atomization pressure: 0.55-1.05 bar; aspirator: 30 M³/h; pump rate: 6-7 ml/min; yield: 151 g; bulk density 0.25 g/ml; tap density: 0.35 g/ml; Angle of repose: 51°. State of amorphous/crystalline: XRPD: amorphous halo; DSC: absence of endothermic peak at 250-258° C.

### Example A4

### Molar Ratio (Meloxicam: HPβCD) of 1:2.5

180.0 g of HPβCD (Kleptose® HPB oral grade) was added to 600 ml water and heated to 70° C. 2 ml ammonia hydroxide solution (28-32%) was then added to this solution. 18 g meloxicam at a molar ratio (meloxicam: HPβCD) of 1:2.5 was added to the solution and fully dissolved. Parameters used for spray drying process: Buchi Mini Spray Dryer B-290; inlet temp: 150° C.; outlet temp: 72-75° C.; atomization pressure: 0.55-1.05 bar; aspirator: 30 M³/h; pump rate: 6-7 ml/min; yield: 100.0 g; bulk density 0.22 g/ml; tap density: 0.33 g/ml; Angle of repose: 48°. State of amorphous/crystalline: XRPD: amorphous halo; DSC: absence of endothermic peak at 250-258° C.

### Example A5

### Molar Ratio (Meloxicam: HPβCD) of 1:1.5

107.0 g of HPβCD (Kleptose® HPB oral grade) was added to 510 ml water and 1 ml ammonia hydroxide solution (28-32%) was then added as well. 18.0 g meloxicam was dissolved using 90 ml acetone, generating a HPβCD solution. The HPβCD solution was mixed with acetone by stirring at room temperature (RT). The molar ratio was (meloxicam: HPβCD): 1:1.5 and fully dissolved. Parameters used for spray drying process: Buchi Mini Spray Dryer B-295; inlet temp: 120° C.; outlet temp: 72-75° C.; atomization pressure: 0.55-1.05 bar; aspirator: 20 M³/h; pump rate: 6-7 ml/min; yield: 100.0 g; bulk density 0.23 g/ml; tap density: 0.31 g/ml; Angle of repose: 48°. State of amorphous/crystalline: XRPD: amorphous halo; DSC: absence of endothermic peak at 250-258° C.

### Example A6

### Molar Ratio (Meloxicam: HPβCD) of 1:2

144.0 g of HPβCD (Kleptose® HPB oral grade) was added to 550 ml water and heated to 75° C. 5 g sodium phosphate was then added to the solution. 18.0 g meloxicam was added to the solution at a_molar ratio of (meloxicam: HPβCD) of 1:2 and then fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M³/h; pump rate: 6-7 ml/min; yield: 151 g; bulk density 0.23 g/ml; tap density: 0.34 g/ml; Angle of repose: 52°. State of amorphous/crystalline: XRPD: amorphous halo; DSC: absence of endothermic peak at 250-258° C.

### Example A7

### Molar Ratio (Meloxicam: HPβCD) of 1:2

144.0 g of HPβCD (Kleptose® HPB oral grade) was added to 500 ml water and heated to 75° C. 1 ml triethylamine was added to the 500 ml solution. 18.0 g meloxicam was added to the solution at a molar ratio (meloxicam: HPβCD) of 1:2 and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M³/h; pump rate: 6-7 ml/min; yield: 151 g; bulk density 0.23 g/ml; tap density: 0.34 g/ml; Angle of repose: 52°. State of amorphous/crystalline: XRPD: amorphous halo; DSC: absence of endothermic peak at 250-258° C.

### Example A8

### Molar Ratio (Meloxicam: HPβCD) of 1:2

72 g of HPβCD was added to 300 ml water and heated to 80° C. 1 ml ethanediamine was then added to the solution. 9 g meloxicam was added to the solution at a molar ratio (meloxicam: HPβCD): 1:2 and fully dissolved. Parameters used for spray drying process: Buchi Mini Spray Dryer B-290; inlet temp: 150° C.; outlet temp: 75-77° C.; atomization pressure: 0.55-1.05 bar; aspirator: 20 M³/h; pump rate: 7-8 ml/min; yield: 76 g; bulk density 0.23 g/ml; tap density: 0.34 g/ml; Angle of repose: 52°. State of amorphous/crystalline: XRPD: amorphous halo; DSC: absence of endothermic peak at 250-258° C.

### Example A9

### Molar Ratio (Meloxicam: SBEβCD) of 1:1.5

86.0 g of SBEβCD (Zibo Qianhui Biological Technology Co.) was added to 450 ml water and heated to 75° C. 2 ml ammonia hydroxide solution (28-32%) was then added to the solution. 14.9 g meloxicam was added to the solution at a molar ratio (meloxicam: SBEβCD) of 1:1.5 and fully dissolved. Parameters used for spray drying process: Buchi Mini Spray Dryer B-290; inlet temp: 140° C.; outlet temp: 80° C.; atomization pressure: 0.55-1.05 bar; aspirator: 32 M³/h; pump rate: 6-7 ml/min; yield: 79 g; bulk density 0.21 g/ml; tap density: 0.33 g/ml; Angle of repose: 50°. State of amorphous/crystalline: XRPD: amorphous halo; DSC: absence of endothermic peak at 250-258° C.

### Example A10

### Molar Ratio (Meloxicam: βCD) of 1:1.5

73.0 g of βCD (CAVAMAX W7) was added to 450 ml water and heated to 75° C. 3 ml ammonia hydroxide solution (28-32%) was then added to the solution. 15.0 g of meloxicam; at a molar ratio (meloxicam: βCD) of 1:1.5 was then added to the solution and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M³/h; pump rate: 6-7 ml/min; yield: 64 g; bulk density 0.21 g/ml; tap density: 0.33 g/ml; Angle of repose: 52°. State of amorphous/crystalline: XRPD: amorphous halo; DSC: absence of endothermic peak at 250-258° C.

### Example A11

### Molar Ratio (Meloxicam: βCD) of 1:2

65.0 g of βCD (CAVAMAX W7) was added to 400 ml water and heated to 75° C. 1.5 ml ammonia hydroxide solution (28-32%) was then added to the solution. 10.0 g meloxicam was added to the solution at a molar ratio (meloxicam: βCD) of 1:2 and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M3/h; pump rate: 6-7 ml/min; yield: 64 g; bulk density 0.23 g/ml; tap density: 0.34 g/ml; Angle of repose: 52°. State of amorphous/crystalline: XRPD: amorphous halo; DSC: absence of endothermic peak at 250-258° C.

### Example A12

### Molar Ratio (Meloxicam: βCD) of 1:2.5

80.0 g of βCD (CAVAMAX W7) was added to 500 ml water and heated to 75° C. 1.5 ml ammonia hydroxide solution (28-32%) was then added to the solution. 10.0 g meloxicam was added at a molar ratio (meloxicam: βCD) of 1:2 to the solution and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M3/h; pump rate: 6-7 ml/min; yield: 64 g; bulk density 0.22 g/ml; tap density: 0.36 g/ml; Angle of repose: 52°. State of amorphous/crystalline: XRPD: amorphous halo; DSC: absence of endothermic peak at 250-258° C.

### Example A13

### Molar Ratio (Meloxicam: HPβCD) of 1:2

144.0 g of HPβCD (Kleptose® HPB oral grade) was added to 500 ml water and heated to 75° C. 5 g L-arginine was then added to the solution. 18.0 g meloxicam was then added to the solution at a molar ratio (meloxicam: HPβCD) of 1:2 and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M³/h; pump rate: 6-7 ml/min; yield: 151 g; bulk density 0.22 g/ml; tap density: 0.33 g/ml; Angle of repose: 52°. State of amorphous/crystalline: XRPD: amorphous halo; DSC: absence of endothermic peak at 250-258° C.

### Example A14

### Molar Ratio (Meloxicam: HPβCD) of 1:2

144.0 g of HPβCD (Kleptose® HPB oral grade) was added to 500 ml water and heated to 75° C. 5 g sodium acetate was added to the solution. 18.0 g meloxicam was then added at a molar ratio (meloxicam: HPβCD) of 1:2 to the solution and fully dissolved. Parameters used for spray drying process: Buchi Mini Spray Dryer B-290; inlet temp: 150° C.; outlet temp: 72-75° C.; atomization pressure: 0.55-1.05 bar; aspirator: 20 M³/h; pump rate: 6-7 ml/min; yield: 151 g; bulk density 0.24 g/ml; tap density: 0.35 g/ml; Angle of repose: 52°. State of amorphous/crystalline: XRPD: amorphous halo; DSC: absence of endothermic peak at 250-258° C.

### Example A15

### Molar Ratio (Meloxicam: HPβCD) of 1:1.5

107.0 g of HPβCD (Kleptose® HPB oral grade) and 1.0 g of polyvinylpyrrolidone K29-32 (PVP K29-32; Ashland) was added to 600 ml water and heated to 70° C. 2.0 ml ammonia hydroxide solution (28-32%) was then added to the solution. 18 g meloxicam was then added to the solution at a molar ratio (meloxicam: HPβCD) of 1:1.5 and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 105.0 g; bulk density 0.27 g/ml; tap density: 0.33 g/ml; Angle of repose: 45°. State of amorphous/crystalline: XRPD: amorphous halo; DSC: absence of endothermic peak at 250-258° C.

### Example A16

### Molar Ratio (Meloxicam: HPβCD) of 1:1.5

107.0 g of HPβCD (Kleptose® HPB oral grade) and 2.0 g of polyethylene glycol 600 (PEG 6000PF; Clariant) was added to 500 ml water and heated to 70° C. 2.0 ml ammonia hydroxide solution (28-32%) was added to this solution. 18 g meloxicam was then added to the solution at a molar ratio (meloxicam: HPβCD) of 1:1.5 and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 90.0 g; bulk density 0.27 g/ml; tap density: 0.33 g/ml; Angle of repose: 46°. State of amorphous/crystalline: XRPD: amorphous halo; DSC: absence of endothermic peak at 250-258° C.

### Example A17

### Molar Ratio (Meloxicam: HPβCD) of 1:1.5

107.0 g of HPβCD (Kleptose® HPB oral grade) and 2.0 g of lecithin (Lipoid) was added to 500 ml water and heated to 70° C. 2.0 ml ammonia hydroxide solution (28-32%) was added to the solution. 18 g meloxicam was added to the solution at a molar ratio (meloxicam: HPβCD) of 1:1.5 and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 115.0 g; bulk density 0.27 g/ml; tap density: 0.33 g/ml; Angle of repose: 47°. State of amorphous/crystalline: XRPD: amorphous halo; DSC: absence of endothermic peak at 250-258° C.

### Example A18

### Molar Ratio (Meloxicam: HPβCD) of 1:1.5

107.0 g of HPβCD (Kleptose® HPB oral grade) and 1.5 g of Gelucire 50/13 (Gattefosse) was added to 400 ml water and heated to 70° C. 2.0 ml ammonia hydroxide solution (28-32%) was then added to the solution. 18 g meloxicam at a molar ratio (meloxicam: HPβCD) of 1:1.5 was added to the solution and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 95.0 g; bulk density 0.27 g/ml; tap density: 0.33 g/ml; Angle of repose: 49°. State of amorphous/crystalline: XRPD: amorphous halo; DSC: absence of endothermic peak at 250-258° C.

### Example A19

### Molar Ratio (Meloxicam: HPβCD) of 1:1.5

107.0 g of HPβCD (Kleptose® HPB oral grade) and 0.5 g of sodium lauryl sulfate was added to 400 ml water and heated to 70° C. 2.0 ml ammonia hydroxide solution (28-32%) was added to the solution. 18 g meloxicam was then added to the solution at a molar ratio (meloxicam: HPβCD) of 1:1.5 and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 95.0 g; bulk density 0.27 g/ml; tap density: 0.33 g/ml; angle of repose: 42°. State of amorphous/crystalline: XRPD: amorphous halo; DSC: absence of endothermic peak at 250-258° C.

### Example A20

### Molar Ratio (Meloxicam: HPβCD) of 1:1.5

107.0 g of HPβCD (Kleptose® HPB oral grade) and 1.0 g of Poloxamer 188 (Nanjing Well Chemical Corp. Ltd) was added to 500 ml water and heated to 70° C. 2.0 ml ammonia hydroxide solution (28-32%) was then added to the solution. 18 g meloxicam at a molar ratio (meloxicam: HPβCD) of 1:1.5 was added to the solution and fully dissolved. Parameters used for spray drying process: Buchi Mini Spray Dryer B-290; inlet temp: 110° C.; outlet temp: 72-75° C.; atomization pressure: 0.55-1.05 bar; aspirator: 30 M³/h; pump rate: 6-7 ml/min; yield: 99.0 g; bulk density 0.27 g/ml; tap density: 0.33 g/ml; angle of repose: 41°. State of amorphous/crystalline: XRPD: amorphous halo; DSC: absence of endothermic peak at 250-258° C.

### Example A21

### Molar Ratio (Meloxicam: HPβCD) of 1:2

144.0 g of HPβCD (Kleptose® HPB oral grade) was added to 500 ml water and was heated to 50° C. 0.8 g sodium hydroxide was then added to the solution. 18.0 g meloxicam at a molar ratio (meloxicam: HPβCD) of 1:2 was added to the solution and fully dissolved. Parameters used for the spray drying process: Buchi Mini Spray Dryer B-290; inlet temp: 150° C.; outlet temp: 80-85° C.; Atomization pressure: 0.55-1.05 bar; aspirator: 30 M³/h; pump rate: 10 ml/min; yield: 145 g; bulk density 0.23 g/ml; tap density: 0.34 g/ml; angle of repose: 52°. State of amorphous/crystalline: XRPD: amorphous halo; DSC: absence of endothermic peak at 250-258° C.

### Example A22

### Molar Ratio (Meloxicam: HPβCD) of 1:2

170.0 g HPβCD (Kleptose® HPB oral grade) was added to 500 ml water and heated to 60° C. 4.0 g meglumine was added to the solution. Then, 21.0 g meloxicam at a_molar ratio (meloxicam: HPβCD) of 1:2 was added to the solution and fully dissolved. Parameters used for the spray drying process: Buchi Mini Spray Dryer B-290; inlet temp: 130° C.; outlet temp: 72-75° C.; Atomization pressure: 0.55-1.05 bar; aspirator: 20 M³/h; pump rate: 5 ml/min; yield: 155 g; bulk density 0.25 g/ml; tap density: 0.36 g/ml; angle of repose: 51°. State of amorphous/crystalline: XRPD: amorphous halo; DSC: absence of endothermic peak at 250-258° C.

### Example A23

### Molar Ratio (Meloxicam: HPβCD) of 1:2

150.0 g HPβCD (Kleptose® HPB oral grade) was added to 500 ml water and heated to 75° C. 2.0 g sodium carbonate was added to the solution. Then, 18.75 g meloxicam at a molar ratio (meloxicam: HPβCD) of 1:2 was added to the solution and fully dissolved. Parameters used for the spray drying process: Buchi Mini Spray Dryer B-290; inlet temp: 140° C.; outlet temp: 75-78° C.; Atomization pressure: 0.55-1.05 bar; aspirator: 25 M³/h; pump rate: 9 ml/min; yield: 155 g; bulk density 0.22 g/ml; tap density: 0.31 g/ml; angle of repose: 50°. State of amorphous/crystalline: XRPD: amorphous halo; DSC: absence of endothermic peak at 250-258° C.

### Example A24

### Molar Ratio (Meloxicam: HPβCD) of 1:2

120.0 g of HPβCD (Kleptose® HPB oral grade) was added to 500 ml water and heated to 80° C. 3.0 g tromethamine was added to the solution. Then, 15.0 g meloxicam at a molar ratio (meloxicam: HPβCD) of 1:2 was added to the solution and fully dissolved. Parameters used for the spray drying process: Buchi Mini Spray Dryer B-290; inlet temp: 110° C.; outlet temp: 60-65° C.; Atomization pressure: 0.55-1.05 bar; aspirator: 28 M³/h; pump rate: 6 ml/min; yield: 110 g; bulk density 0.24 g/ml; tap density: 0.29 g/ml; angle of repose: 50°. State of amorphous/crystalline: XRPD: amorphous halo; DSC: absence of endothermic peak at 250-258°C.

### Examples B1-B14

### Experimental Methods for Preparing Formulations Including Meloxicam-Cyclodextrin Inclusion Complex

The formulations examples listed below include one or more or all of the following manufacturing processes. The examples are intended to illustrate and not to limit the formulation manufacturing process as well as the formulation compositions.
(1) Generating a blend: weigh pharmaceutical excipients and sieve each individually (1 mm); for excipient of small quantity, pre-mixing or dilution is required.
(2) Dry granulation: load the above blend to a roller compactor (e.g.: Zhang Jiagang Creation Mechanical Manufacturing Co., Model: GL2-25). The experimental parameters are listed as follows: gap (mm): 0.11-0.22; roll speed (rpm): 5-6 rpm, feed speed: 7-9 rpm. Screen size: 0.8-1.2 mm. Characterization of the granules are as follows: bulk density: 0.40-0.45 g/ml; tap density: 0.53-0.60 g/ml; angle of repose 30-38°: The yield of the granule is 80-95%.
(3) Final blend: based on the amount of the granule, calculate the amount of the magnesium stearate. Add the magnesium stearate to the above granule for the final blend 100 revolutions (4 min, 25 rpm).
(4) Encapsulation: depending on the fill weight, fill the above blend into “Size 4” or “Size 3” or “Size 2” opaque hard-gelatin capsules (Manufacturer: Capsugel Suzhou, China). For example, for a fill-weight of 112 mg that uses “Size 3”: the capsule shell is approx. 46 mg; the variation for the target fill weight is <5%, i.e., 106.4 mg to 117.6 mg per capsule unit, and for the total capsule weight, 152.4 mg to 163.6 mg per unit. (5) Packaging: the capsules are placed in 60cc HDPE bottles (50 units/bottle), followed by aluminum seal.

### Example B1

### Meloxicam-HPβCD Capsule 6 mg

| Ingredient | mg/unit | % (w/w) |
|---|---|---|
| Intermediate (A2) | 41.67 | 50.00 |
| Microcrystalline cellulose | 33.34 | 40.00 |
| Lactose monohydrate | 4.17 | 5.00 |
| Crospovidone | 3.34 | 4.00 |
| Magnesium stearate | 0.83 | 1.00 |
| Total | 83.35 | 100.00 |

### Example B2

### Meloxicam-HPβCD Capsule 7.5 mg

| Ingredient | mg/unit | % (w/w) |
|---|---|---|
| Intermediate (A3) | 67.50 | 50.00 |
| Microcrystalline cellulose | 54.00 | 40.00 |
| Lactose monohydrate | 8.10 | 6.00 |
| Crospovidone | 4.05 | 3.00 |
| Magnesium stearate | 1.35 | 1.00 |
| Total | 135.00 | 100.00 |

### Example B3

### Meloxicam-HPβCD Capsule 5 mg

| Ingredient | mg/unit | % (w/w) |
|---|---|---|
| Intermediate (A4) | 55.00 | 50.00 |
| Microcrystalline cellulose | 44.00 | 40.00 |
| Lactose monohydrate | 6.60 | 6.00 |
| Crospovidone | 3.30 | 3.00 |
| Magnesium stearate | 1.10 | 1.00 |
| Total | 110.00 | 100.00 |

### Example B4

### Meloxicam-HPβCD Capsule 7.5 mg

| Ingredient | mg/unit | % (w/w) |
|---|---|---|
| Intermediate (A5) | 52.00 | 50.00 |
| Microcrystalline cellulose | 41.60 | 40.00 |
| Lactose monohydrate | 5.20 | 5.00 |
| Crospovidone | 4.06 | 4.00 |
| sodium bisulfite | 0.10 | 0.10 |
| Magnesium stearate | 1.04 | 1.00 |
| Total | 104.00 | 100.00 |

### Example B5

### Meloxicam-HPβCD Capsule 5 mg

| Ingredient | mg/unit | % (w/w) |
|---|---|---|
| Intermediate (A6) | 46.40 | 50.00 |
| Microcrystalline cellulose | 37.22 | 40.00 |
| Lactose monohydrate | 5.66 | 6.00 |
| Crospovidone | 2.78 | 3.00 |
| Magnesium stearate | 0.94 | 1.00 |
| Total | 93.00 | 100.00 |

### Example B6

### Meloxicam-βCD Capsule 7.5 mg

| Ingredient | mg/unit | % (w/w) |
|---|---|---|
| Intermediate (A10) | 44.00 | 50.00 |
| Microcrystalline cellulose | 35.20 | 40.00 |
| Lactose monohydrate | 5.28 | 6.00 |
| Crospovidone | 2.64 | 3.00 |
| Magnesium stearate | 0.88 | 1.00 |
| Total | 88.00 | 100.00 |

### Example B7

### Meloxicam-SBEβCD Capsule 7.5 mg

| Ingredient | mg/unit | % (w/w) |
|---|---|---|
| Intermediate (A9) | 50.50 | 50.00 |
| Microcrystalline cellulose | 40.40 | 40.00 |
| Lactose monohydrate | 6.06 | 6.00 |
| Croscarmellose sodium | 3.03 | 3.00 |
| Magnesium stearate | 1.01 | 1.00 |
| Total | 101.00 | 100.00 |

### Example B8

### Meloxicam-HPβCD Capsule 7.5 mg

| Ingredient | mg/unit | % (w/w) |
|---|---|---|
| Intermediate (A5) | 52.00 | 50.00 |
| Microcrystalline cellulose | 41.60 | 40.00 |
| Lactose monohydrate | 6.24 | 6.00 |
| Sodium starch glycolate | 3.12 | 3.00 |
| Magnesium stearate | 1.04 | 1.00 |
| Total | 104.00 | 100.00 |

### Example B9

### Meloxicam-HPβCD Capsule 7.5 mg

| Ingredient | mg/unit | % (w/w) |
|---|---|---|
| Intermediate (A5) | 52.00 | 50.00 |
| Microcrystalline cellulose | 41.60 | 40.00 |
| Mannitol | 5.72 | 5.50 |
| Ascorbic acid | 0.52 | 0.5 |
| Sodium starch glycolate | 3.12 | 3.00 |
| Magnesium stearate | 1.04 | 1.00 |
| Total | 104.00 | 100.00 |

### Example B10

### Meloxicam-HPβCD Capsule 12 mg

| Ingredient | mg/unit | % (w/w) |
|---|---|---|
| Intermediate (A6) | 111.36 | 46.40 |
| Ascorbic acid | 0.48 | 0.20 |
| Lactose monohydrate | 118.56 | 49.40 |
| Sodium starch glycolate | 7.20 | 3.00 |
| Talc | 2.40 | 1.00 |
| Total | 240.00 | 100.00 |

### Example B11

### Meloxicam-HPβCD Capsule 12 mg

| Ingredient | mg/unit | % (w/w) |
|---|---|---|
| Intermediate (A6) | 111.36 | 46.40 |
| Butylated hydroxytoluene | 0.48 | 0.20 |
| Lactose anhydrous | 118.56 | 49.40 |
| croscarmellose sodium | 7.20 | 3.00 |
| Silicon dioxide | 2.40 | 1.00 |
| Total | 240.00 | 100.00 |

### Example B12

### Meloxicam-HPβCD Capsule 12 mg

| Ingredient | mg/unit | % (w/w) |
|---|---|---|
| Intermediate (A6) | 111.36 | 46.40 |
| Racemethionine | 0.48 | 0.20 |
| Starch | 118.56 | 49.40 |
| Crospovidone | 7.20 | 3.00 |
| Magnesium stearate | 2.40 | 1.00 |
| Total | 240.00 | 100.00 |

### Example B13

### Meloxicam-HPβCD Capsule 5.5 mg

| Ingredient | mg/unit | % (w/w) |
|---|---|---|
| Intermediate (A6) | 51.04 | 46.40 |
| Butylated Hydroxytoluene | 0.22 | 0.20 |
| Mannitol | 54.34 | 49.40 |
| croscarmellose sodium | 3.30 | 3.00 |
| Silicon dioxide | 1.10 | 1.00 |
| Total | 110.00 | 100.00 |

### Example B14

### Meloxicam-HPβCD Capsule 5.5 mg

| Ingredient | mg/unit | % (w/w) |
|---|---|---|
| Intermediate (A6) | 51.04 | 46.40 |
| Racemethionine | 0.22 | 0.20 |
| Microcrystalline cellulose | 54.34 | 49.40 |
| Crospovidone | 3.30 | 3.00 |
| Magnesium Stearate | 1.10 | 1.00 |
| Total | 110.00 | 100.00 |

TABLE A
Illustrative Dissolution Data for Meloxicam in Various Forms

| Sample description | Capsule # | 5 min | 10 min | 20 min | 30 min | 60 min |
|---|---|---|---|---|---|---|
| Intermediate: Meloxicam- | 1 | 52.9 | 97.2 | 99.1 | 99.2 | 99.1 |
| HPβCD inclusion complex; molar | 2 | 71.6 | 95.1 | 98.6 | 100.0 | 98.9 |
| ratio: 1:2; hard- | 3 | 61.6 | 94.2 | 99.3 | 101.2 | 99.3 |
| gelatin capsule; 7.5 mg | Average | 62.0 | 95.5 | 99.0 | 100.2 | 99.1 |
| | SD | 7.6 | 1.3 | 0.3 | 0.8 | 0.2 |
| | RSD (%) | 12.3 | 1.3 | 0.3 | 0.8 | 0.2 |
| PM (physical mixture) Meloxicam- | 1 | 0.7 | 0.5 | 6.1 | 9.7 | 15.2 |
| HPβCD physical mixture; molar | 2 | 0.6 | 1.7 | 7 | 11.1 | 14.6 |
| ratio: 1:2; hard- | 3 | 0.5 | 2.2 | 8.5 | 13.3 | 20.2 |
| gelatin capsule; 7.5 mg | Average | 0.6 | 1.5 | 7.2 | 11.4 | 16.7 |
| | SD | 0.1 | 0.7 | 1.0 | 1.5 | 2.5 |
| | RSD (%) | 13.6 | 48.6 | 13.7 | 13.0 | 15.1 |
| Formulation including Intermediate, | 1 | 52.9 | 92.0 | 94.2 | 96.7 | 97.8 |
| or Meloxicam-HPβCD inclusion | 2 | 71.6 | 100.3 | 101.6 | 102.5 | 102.9 |
| complex; molar ratio: hard-gelatin | 3 | 81.3 | 99.2 | 100.6 | 101.0 | 100.6 |
| capsule; 7.5 mg | Average | 68.6 | 97.2 | 98.8 | 100.1 | 100.4 |
| | SD | 14.4 | 4.5 | 4.0 | 3.0 | 2.6 |
| | RSD (%) | 21.0 | 4.6 | 4.0 | 3.0 | 2.6 |
| Intermediate: Meloxicam- | 1 | 46.4 | 92.5 | 98.9 | 99.8 | 99.4 |
| βCD inclusion complex; molar | 2 | 42.6 | 93.2 | 100.1 | 101.7 | 101.2 |
| ratio: 1:2; hard-gelatin | 3 | 48.7 | 94.0 | 99.1 | 100.9 | 100.5 |
| capsule; 7.5 mg | Average | 45.9 | 93.2 | 99.4 | 100.8 | 100.4 |
| | SD | 3.1 | 0.8 | 0.6 | 1.0 | 0.9 |
| | RSD (%) | 6.7 | 0.8 | 0.6 | 0.9 | 0.9 |
| PM (physical mixture) Meloxicam- | 1 | 0.7 | 0.5 | 5.4 | 9.7 | 17.1 |
| βCD physical mixture; molar | 2 | 0.7 | 1.8 | 7.0 | 10.8 | 15.8 |
| ratio: 1:2 hard- | 3 | 0.5 | 2.3 | 9.7 | 14.6 | 21.6 |
| gelatin capsule; 7.5 mg | Average | 0.6 | 1.5 | 7.4 | 11.7 | 18.2 |
| | SD | 0.1 | 0.9 | 2.2 | 2.6 | 3.1 |
| | RSD (%) | 21.7 | 62.4 | 29.4 | 22.2 | 16.9 |
| Formulation including | 1 | 70.9 | 95.9 | 97.6 | 98.1 | 99.2 |
| Intermediate, or Meloxicam-βCD | 2 | 88.6 | 99.0 | 100.8 | 101.1 | 102.2 |
| inclusion complex; molar | 3 | 81.1 | 95.3 | 97.1 | 97.4 | 998.3 |
| ratio: 1:2; hard-gelatin | Average | 80.2 | 96.6 | 98.5 | 98.8 | 99.9 |
| capsule; 7.5 mg | SD | 8.8 | 2.1 | 2.0 | 2.0 | 2.1 |
| | RSD (%) | | 2.1 | 2.1 | 2.0 | 2.2 |
| Mobic ® Tablet 7.5 mg | 1 | 8.4 | 18.7 | 28.3 | 34.9 | 47.2 |
| | 2 | 10.5 | 19.9 | 30.0 | 35.7 | 47.4 |
| | 3 | 7.8 | 16.6 | 26.5 | 31.6 | 44.1 |
| | Average | 8.9 | 18.4 | 28.3 | 34.1 | 46.2 |
| | SD | 1.4 | 1.7 | 1.8 | 2.2 | 1.9 |
| | RSD (%) | 15.9 | 9.1 | 6.2 | 6.4 | 4.0 |

*Dissolution test condition: medium: pH 6.1 (0.05M phosphate buffer), basket method, 100 rpm, 900 ml, 37° C.

### Examples C1-C2-C3

### Animal (Dog) Pharmacokinetic Study

### Example C1

### Comparative Pharmacokinetic Study of Meloxicam Formulations Following Single Oral Administrations to Non-Naïve Beagle Dogs

Test System and Study Design:

Two male and two female non-naïve beagle dogs with body weight over the range of 8.62-9.40 kg were assigned to this study. Each animal had a unique skin tattoo number on ear as the identification. The four dogs were orally dosed with the meloxicam formulations in the treatment sequence shown in the Table B. Blood samples were harvested according to each sampling time.

TABLE B

| | | | | | Strength of the | | | |
|---|---|---|---|---|---|---|---|---|
| | | | | | Formulation | | Nominal | |
| Group/ | # of | # of | | Dose | (mg/capsule or tablet | | Dose Level | Dose |
| Phase | Males | Females | Test Formulations | Route | or 5 mL of water) | Vehicle | (mg/kg) | Quantities |
| 1/1 | 2 | 2 | Meloxicam | oral | 7.5 | — | 0.75 | 1 capsule/dog |
| | | | Formulation | | | | | |
| | | | (Meloxicam-βCD | | | | | |
| | | | Capsule 7.5 mg) | | | | | |
| 1/2 | 2 | 2 | Mobic ® | oral | 7.5 | — | 0.75 | 1 tablet/dog |
| | | | (Tablet 7.5 mg) | | | | | |
| 1/3 | 2 | 2 | Meloxicam | oral | 7.5 | 5 mL | 0.75 | — |
| | | | Formulation | | | of | | |
| | | | (Meloxicam-βCD | | | water | | |
| | | | Suspension 7.5 mg) | | | | | |

Dose Preparation and Administration:

In Phase 1 and Phase 2, the Meloxicam Formulation (Meloxicam-βCD Capsule 7.5 mg) and Mobic® (Tablet 7.5 mg) were dosed, respectively. In Phase 3, Meloxicam Formulation (Meloxicam-βCD Suspension 7.5 mg) was dosed. The preparation procedure for the suspension is as follows: opened one capsule shell, emptied the powder content to a vial containing 5 mL water. Stirred the resultant mixture between 30 seconds to 1 minute and dosed the animal immediately. Administered the suspension to the dogs which was followed by 6 mL of water to fully wash out the leftover powder in the vials.

Animals were weighed prior to dose administration and the body weights were recorded on the dose record sheets.

Sample Collection and Preparation:

Serial blood samples (approximately 0.5 mL in K₂EDTA) were collected via a cephalic vein. Blood samples were collected at pre-dose and at 0.25, 0.5, 0.75, 1, 1.25, 1.5, 2, 3, 4, 6, 8, 12, 24 and 48 hours post-dose for the three phases.

Dog plasma samples were analyzed for Meloxicam using a qualified bio-analytical method based on protein precipitation followed by HPLC/MS/MS analysis.

The lower limit of quantification (LLOQ) for Meloxicam in plasma was 3.0 ng/mL and the upper limit of quantification (ULOQ) in plasma was 3000 ng/mL.

Pharmacokinetics Data Analysis:

Plasma concentration data of Meloxicam were subjected to a non-compartmental pharmacokinetic analysis using WinNonlin™ Version 6.2.1 (Pharsight, Mountain View, Calif.). Peak plasma concentrations (Cₘₐₓ) and the corresponding peak times (Tₘₐₓ) were taken directly from the plasma concentration versus time profiles.

Terminal half-life (t_{1/2}), mean residence time (MRT) from time zero to infinity (MRT_{0-inf}), mean residence time (MRT) from time zero to the last quantifiable concentration (MRT₀₋ₗₐₛₜ), the area under the plasma concentration time curve (AUC) from time zero to the last quantifiable concentration (AUC₀₋ₗₐₛₜ) and AUC from time zero extrapolated to infinity (AUC_{0-inf}) were calculated using the model of linear log trapezoidal.

MRT, t_{1/2} and Tₘₐₓ values were reported to two decimal places. Other PK parameters such as AUC and Cₘₐₓ values were reported to three significant figures.

Nominal sampling times were used to calculate all pharmacokinetic parameters since in no situations were there a deviation larger than 5% between the actual and nominal sampling times.

Pharmacokinetics

Following single oral dosing of Meloxicam formulations to non-naïve male and female beagle dogs at 7.5 mg per dog, the mean values of main pharmacokinetic parameters such as Cₘₐₓ, Tₘₐₓ, AUC₀₋ₗₐₛₜ, AUC_{0-inf}, AUC_% Extrap_obs, t_{1/2}, MRT₀₋ₗₐₛₜ, MRT_{0-inf} are presented in

Table C (as a non-limiting example, see FIG. 15 for illustration).

TABLE C

| | | | Meloxicam |
|---|---|---|---|
| | Meloxicam | | Formulation |
| | Formulation | Mobic ® | (Meloxicam-βCD |
| Pharmacokinetics | (Meloxicam-βCD | (Tablet | Suspension |
| Parameters | Capsule 7.5 mg) | 7.5 mg) | 7.5 mg) |
| Cₘₐₓ (ng/mL) | 3470 | 2990 | 3480 |
| Tₘₐₓ (h) | 2.00 | 4.00 | 1.50 |
| T_{1/2} (h) | 25.03 | 34.00 | 28.03 |
| AUC₀₋ₗₐₛₜ (ng/mL · hr) | 82000 | 73500 | 85300 |
| AUC_{0-inf} (ng/mL · hr) | 110000 | 115000 | 125000 |
| MRT₀₋ₗₐₛₜ (h) | 19.80 | 20.89 | 20.06 |
| MRT_{0-inf} (h) | 36.39 | 48.33 | 41.96 |
| AUC_% Extrap_obs | 25.8 | 36.0 | 32.0 |
| Relative | 112 | — | 116 |
| Bioavailability (%) | | | |

Study Summary:

Meloxicam formulation including meloxicam-βCD inclusion complex (Meloxicam Formulation Capsule 7.5 mg, or suspension 7.5 mg) was dosed at 0.75 mg/kg to male and female beagle dogs via oral administration.

The pharmacokinetics of both present Meloxicam Formulations (Capsule 7.5 mg, Suspension 7.5 mg) demonstrated a rapid absorption with a time to peak plasma concentration (Tₘₐₓ) 1.5-2 hrs (capsule: 2 hrs; suspension: 1.5 hrs), as compared to Mobic® (tablet) (Tₘₐₓ: 4 hrs). The Cₘₐₓ was slightly increased for Meloxicam Formulations as opposed to the Mobic®, while the AUC remained largely unchanged between present Meloxicam Formulations and

The relative oral bioavailability of the present Meloxicam Formulations (Capsule 7.5 mg, Suspension 7.5 mg) to Mobic® (Tablet 7.5 mg) was 112% and 116%, respectively. This demonstrates that the present Meloxicam Formulation (capsule or suspension) has an improved absorption as compared to Mobic®.

The pharmacokinetic profiles of the present Meloxicam Formulations (Capsule 7.5 mg, Suspension 7.5 mg) show no significant different between each other, though the suspension has a slight edge in Tₘₐₓ (1.5 hrs versus 2 hrs). It is likely that capsules formulation may experience a slow capsule shell collapsing in vivo. It is noted that the systematic exposure (Cₘₐₓ and AUC) of the drug from these two formulations are comparable.

No marked sex differences in systemic exposure (Cₘₐₓ and AUC₀₋ₗₐₛₜ) and Tₘₐₓ were observed at the given dosage for the present Meloxicam Formulations (Capsule 7.5 mg, Suspension 7.5 mg) and for Mobic® (tablet).

No adverse effects were observed during and after dosing for all the study dogs after receiving single oral administration of the present Meloxicam Formulations (Capsule 7.5 mg, Suspension 7.5 mg) at 7.5 mg/dog, indicating the safety of the present Meloxicam Formulations (Capsule 7.5 mg, Suspension 7.5 mg). The same result was obtained using Mobic®.

FIG. 15 illustrates a comparison of plasma concentrations in non-naive beagle dogs after oral administration with of Meloxicam formulations (capsule) and Mobic® at 7.5 mg per dog.

### Example C2

### Comparative Pharmacokinetic Study of Meloxicam Formulations (Capsules) and Mobic® Tablet 7.5 mg Following Single Oral Administrations to Non-Naïve Beagle Dogs

A second round of comparative pharmacokinetic evaluation was performed using the inventive compositions including the Meloxicam-cyclodextrin formulations in different compositions (Meloxicam-βCD formulation, Meloxicam-HPβCD formulation) and varying strengths (Meloxicam-HPβCD formulations at different strengths) as compared to the Meloxicam commercial product Mobic® (Boehringer Ingelheim Pharmaceuticals).

Test System and Study Design:

In Phase 1, two male and two female beagle dogs with body weight over the range of 9.53-10.66 kg were assigned to this study. These four beagle dogs were assigned to the following four phases at the same dosing order. Each animal had a unique skin tattoo number on ear as the identification. The four dogs were orally dosed with the Meloxicam formulations in the treatment sequence of Meloxicam-βCD Formulation (capsule 7.5 mg)-Meloxicam-HPβCD Formulation (capsule 7.5 mg)-Meloxicam-HPβCD Formulation (capsule 6.0 mg)-Mobic® (tablet 7.5 mg)-Meloxicam-HPβCD Formulation (capsule 5.0 mg). Blood samples were harvested according to each sampling time-point. Results are shown in Table D:

TABLE D

| | | | | | Strength | Nominal | |
|---|---|---|---|---|---|---|---|
| Group/ | # of | # of | Test | Dose | (mg/capsule | Dose Level | Dose |
| Phase | Males | Females | Formulations | Route | or tablet) | (mg/kg) | Quantity |
| 1/1 | 2 | 2 | Meloxicam-βCD | oral | 7.5 | 0.75 | 1 capsule/dog |
| | | | Formulation | | | | |
| | | | (capsule 7.5 mg) | | | | |
| 1/2 | 2 | 2 | Meloxicam-HPβCD | oral | 7.5 | 0.75 | 1 capsule/dog |
| | | | Formulation | | | | |
| | | | (capsule 7.5 mg) | | | | |
| 1/3 | 2 | 2 | Meloxicam-HPβCD | oral | 6.0 | 0.60 | 1 capsule/dog |
| | | | Formulation | | | | |
| | | | (capsule 6.0 mg) | | | | |
| 1/4 | 2 | 2 | Mobic ® (tablet, | oral | 7.5 | 0.75 | 1 tablet/dog |
| | | | product; 7.5 mg) | | | | |
| 1/5 | 2 | 2 | Meloxicam-HPβCD | oral | 5.0 | 0.50 | 1 capsule/dog |
| | | | Formulation | | | | |
| | | | (capsule 5.0 mg) | | | | |

Sample Collection and Preparation:

Serial blood samples (approximately 0.5 mL in K₂EDTA) were collected via a peripheral vessel. Blood samples were collected at pre-dose and at 0.25, 0.5, 0.75, 1, 1.25, 1.5, 2, 3, 4, 6, 8, 12, 24 and 48 hours post-dose for the five phases.

After collection, blood samples were gently inverted several times and immediately placed on wet ice prior to centrifugation at 2 to 8° C. and 3000 g for 10 minutes. At least 0.2 mL plasma was harvested and transferred into a pre labeled transparent vial, and stored frozen at 60° C. or lower until shipped on dry ice for analysis.

Sample Analysis:

Dog plasma samples were analyzed for Meloxicam using a qualified bioanalytical method based on protein precipitation followed by LC/MS/MS analysis.

The lower limit of quantification (LLOQ) for Meloxicam in plasma was 1.00 ng/mL and the upper limit of quantification (ULOQ) in plasma was 3000 ng/mL for each phase.

Pharmacokinetic Data Analysis:

Plasma concentration data of Meloxicam were subjected to a non-compartmental pharmacokinetic analysis using WinNonlin Version 6.2.1 (Pharsight, Mountain View, Calif.). Peak plasma concentrations (Cₘₐₓ) and the corresponding peak times (Tₘₐₓ) were taken directly from the plasma concentration versus time profiles.

Terminal half-life (t_{1/2}), mean residence time (MRT) from time zero to infinity (MRT_{0-inf}), mean residence time (MRT) from time zero to the last quantifiable concentration (MRT₀₋ₗₐₛₜ) the area under the plasma concentration time curve (AUC) from time zero to the last quantifiable concentration (AUC₀₋ₗₐₛₜ) and AUC from time zero extrapolated to infinity (AUC_{0-inf}) were calculated using the model of linear log trapezoidal.

MRT, t_{1/2} and Tₘₐₓ values were reported to two decimal places. Other PK parameters such as AUC and Cₘₐₓ values were reported to three significant figures.

Nominal sampling times were used to calculate all pharmacokinetic parameters since there was no deviation larger than 5% between the actual and nominal sampling times.

Following single oral dosing of Meloxicam formulations to non-naïve male and female beagle dogs, the mean values of main pharmacokinetic parameters such as Cₘₐₓ, Tₘₐₓ, AUC₀₋ₗₐₛₜ, AUC_{0-inf}, AUC_% Extrap_obs, t_{1/2}, MRT₀₋ₗₐₛₜ, MRT_{0-inf} are presented below in Table E (as a non-limiting example, see FIG. 16 for illustration).

TABLE E

| | Meloxicam formulations (in capsules) | | | | |
|---|---|---|---|---|---|
| | | | Meloxicam- | Meloxicam- | |
| | | Meloxicam- | HPβCD | HPβCD | |
| | Meloxicam-βCD | HPβCD | Formulation | Formulation | Mobic ® |
| Pharmacokinetic | Formulation | Formulation | (Capsule | (Capsule | (in Tablet, |
| Parameters | (Capsule 7.5 mg) | (Capsule 7.5 mg) | 6.0 mg) | 5.0 mg) | 7.5 mg) |
| Number of time points used | 5 | 10 | 8 | 12 | 7 |
| for calculation of t_{1/2} | | | | | |
| Cₘₐₓ (ng/mL) | 3440 | 3940 | 3150 | 2690 | 2730 |
| Tₘₐₓ (h) | 1.50 | 1.00 | 1.50 | 0.50 | 2.00 |
| t_{1/2} (h) | 36.18 | 24.50 | 30.96 | 25.31 | 31.83 |
| AUC₀₋ₗₐₛₜ (ng/mL · hr) | 79200 | 93600 | 80700 | 57200 | 73000 |
| AUC_{0-inf} (ng/mL · hr) | 126000 | 127000 | 122000 | 80400 | 108000 |
| MRT₀₋ₗₐₛₜ (h) | 20.21 | 19.70 | 20.64 | 20.20 | 20.78 |
| MRT_{0-inf} (h) | 50.07 | 36.29 | 45.02 | 38.80 | 44.66 |
| AUC_% Extrap_obs | 37.3 | 26.1 | 33.8 | 28.9 | 32.7 |
| Relative | 108 | 128 | 111 | 78.4 | NA |
| Bioavailability (%) | | | | | |

NA: Not applicable
Relative bioavailability of Meloxicam βCD and HPβCD capsules to commercial tablets were calculated without taking the dosage form into consideration.

Study Summary:

The present Meloxicam formulations were dosed at 5.0, 6.0 to 7.5 mg/capsule/dog, respectively, to two male and two female beagle dogs via oral administration. These formulations were comprised of meloxicam-βCD or HPβCD inclusion complex. Mobic®, the meloxicam commercial product, was also dosed at 7.5 mg/tablet/dog to the same four beagle dogs. The dosing was arranged in the sequence as follows: 1st Phase: Meloxicam-βCD Formulation (7.5 mg); 2nd Phase: Meloxicam-HPβCD Formulation (7.5 mg); 3rd Phase: Meloxicam-HPβCD Formulation (6.0 mg); 4th Phase: Mobic® (Tablet, 7.5 mg); 5th Phase: Meloxicam-HPβCD Formulation (5.0 mg). There was at least one week washout period between each phase.

The pharmacokinetics profiles of Meloxicam-βCD Formulation (capsule 7.5 mg) and Meloxicam HPβCD Formulations (capsules, 7.5 mg, 6.0 mg and 5.0 mg) all show faster time-to-plasma-peak-concentration with a Tₘₐₓ at the range of 0.5 to 1.5 hour, as compared to Mobic® (tablet) with a Tₘₐₓ at 2.0 hours. It is also noted that Meloxicam-HPβCD Formulations generally show an even faster Tₘₐₓ at 0.5-1.0 hour, as compared to Meloxicam-βCD Formulation (7.5 mg) which has a Tₘₐₓ at 1.5 hour.

The pharmacokinetics profiles of both Meloxicam-βCD Formulation and Meloxicam HPβCD Formulations show a greater systemic exposure (AUC) and plasma peak concentration (Cₘₐₓ), as compared to those of Mobic®. It is also noted that Meloxicam HPβCD Formulations show greater AUC and Cₘₐₓ than those of the Meloxicam-βCD Formulation. For example, the Cₘₐₓ for Meloxicam-HPβCD, Meloxicam-βCD and Mobic®, all at 7.5 mg level are: 3940, 3440 and 2730 ng/mL, respectively; the exposure AUC0 last in the same sequence, 93600, 79200, 73000 ng/mL·hr.

Further, both exposure (AUC) and Cₘₐₓ increase as the drug strength in Meloxicam-HPβCD Formulations increases from 5.0 to 6.0, to 7.5 mg/dog. The Cₘₐₓ are 2690, 3150, 3940 ng/mL, respectively; the exposure, in the same sequence: 57200, 80700, 93600 ng/mL·hr in AUC₀₋ₗₐₛₜ.

The above results seem to suggest that, out of all the meloxicam formulations, the exposure (AUC) and Cₘₐₓ of Meloxicam-HPβCD Formulations 6.0 mg and 5.0 mg are more comparable to those of Mobic® (7.5 mg). The Cₘₐₓ and AUC₀₋ₗₐₛₜ ratios of Meloxicam HPβCD Formulation (5.0 mg) to Mobic® are 0.985 and 0.784 respectively. The Cₘₐₓ and AUC₀₋ₗₐₛₜ ratios of Meloxicam HPβCD Formulation (6.0 mg) to Mobic® (7.5 mg) are 1.15 and 1.111, respectively.

A spike of the plasma concentration at the 24th hour was noted. Without wishing to be bound by theory, it is believed that entero-hepatic and/or entro-enteric circulation may contribute to this spike which results in a prolonged half-life (t_{1/2}).

No marked sex differences in systemic exposure (Cₘₐₓ and AUC₀₋ₗₐₛₜ) and Tₘₐₓ were observed at the given dosage for the present Meloxicam Formulations and Mobic®.

No adverse effects were observed during and after dosing for all the study dogs after receiving single oral administration of the present Meloxicam Formulations, indicating the safety of the present Meloxicam Formulations. The same result is obtained with Mobic®.

### Example C3

### A Third Round of Comparative Pharmacokinetic and Dose Proportionality Study of a Meloxicam-HPβCD Formulation Following Single Oral Administrations to Non-Naïve Beagle Dogs

The objectives of this study were to determine the pharmacokinetic profiles and dose proportionality of the meloxicam-HPβCD formulation, manufactured by WuXi AppTec Co., at different dose levels (5.5 mg or 11.0 mg), as compared with Mobic® tablet 7.5 mg (meloxicam commercial product, manufactured by Boehringer Ingelheim Pharmaceuticals), following single oral (PO) doses in male and female non-naive beagle dogs. The meloxicam concentration was monitored in plasma for up to 48 hours.

Test System and Study Design:

Two male and two female beagle dogs with body weights over the range of 9.48-10.39 kg were assigned to this study. These same four beagle dogs were assigned to each of the following three phases at the same dosing order. Each animal had a unique skin tattoo number on ear as the identification. The four dogs were orally dosed with meloxicam formulations in the treatment sequence of meloxicam-HPβCD Formulation Capsule at 5.5 mg (1 capsule per dog), Mobic® 7.5 mg (1 tablet per dog), and meloxicam-HPβCD Formulation Capsule at 11.0 mg (2 capsules per dog). Blood samples were harvested according to each sampling time point as in Table F.

TABLE F

| Group/ | # of | # of | | Dose | | Nominal Dose Level | Dose |
|---|---|---|---|---|---|---|---|
| Phase | Males | Females | Test Formulations | Route | Dose per dog | (mg/kg) | Quantitiy |
| 1/1 | 2 | 2 | Meloxicam-HPβCD Formulation | oral | 5.5 mg | 0.55 | 1 capsule/dog |
| | | | Capsule 5.5 mg | | | | |
| | | | Molar ratio | | | | |
| | | | (meloxicam: HPβCD) = 1:2 | | | | |
| 1/2 | 2 | 2 | Mobic ® 7.5 mg | oral | 7.5 mg | 0.75 | 1 tablet/dog |
| | | | (meloxicam commercial product) | | | | |
| 1/3 | 2 | 2 | Meloxicam-HPβCD Formulation | oral | 11.0 mg | 1.1 | 2 capsules/dog |
| | | | Capsule 5.5 mg | | | | |
| | | | Molar ratio | | | | |
| | | | (meloxicam: HPβCD) = 1:2 | | | | |

Sample Collection and Preparation:

Serial blood samples (approximately 0.5 mL in K₂EDTA) were collected via a peripheral vessel. Blood samples were collected at pre-dose and at 0.25, 0.5, 0.75, 1, 1.25, 1.5, 2, 3, 4, 6, 8, 12, 24 and 48 hours post-dose for the five phases.

After collection, blood samples were gently inverted several times and immediately placed on wet ice prior to centrifugation at 2 to 8° C. and 3000 g for 10 minutes. At least 0.2 mL plasma was harvested and transferred into a pre labeled transparent vial, and stored frozen at 60° C. or lower until shipped on dry ice for analysis.

Sample Analysis:

Dog plasma samples were analyzed for Meloxicam using a qualified bioanalytical method based on protein precipitation followed by LC/MS/MS analysis.

The lower limit of quantification (LLOQ) for Meloxicam in plasma was 1.00 ng/mL and the upper limit of quantification (ULOQ) in plasma was 3000 ng/mL for each phase.

Pharmacokinetic Data Analysis:

Plasma concentration data of Meloxicam were subject to a non-compartmental pharmacokinetic analysis using WinNonlin Version 6.2.1 (Pharsight, Mountain View, Calif.). Peak plasma concentrations (Cₘₐₓ) and the corresponding peak times (Tₘₐₓ) were taken directly from the plasma concentration versus time profiles.

Terminal half-life (t_{1/2}), mean residence time (MRT) from time zero to infinity (MRT_{0-inf}), mean residence time (MRT) from time zero to the last quantifiable concentration (MRT₀₋ₗₐₛₜ), the area under the plasma concentration time curve (AUC) from time zero to the last quantifiable concentration (AUC₀₋ₗₐₛₜ) and AUC from time zero extrapolated to infinity (AUC_{0-inf}) were calculated using the model of linear log trapezoidal. MRT, t_{1/2} and Tₘₐₓ values were reported to two decimal places. Other PK parameters such as AUC and Cₘₐₓ values were reported to three significant figures.

Nominal sampling times were used to calculate all pharmacokinetic parameters since there was no deviation larger than 5% between the actual and nominal sampling times.

Following single oral dosing of meloxicam-HPβCD formulation (which contain the meloxicam-HPβCD inclusion complex with a molar ratio of 1:2) and Mobic® (meloxicam commercial product) to non-naïve male and female beagle dogs, the mean values of main pharmacokinetic parameters such as Cₘₐₓ, Tₘₐₓ, AUC₀₋ₗₐₛₜ, AUC_{0-inf}, AUC_{Extrap}_{_}_{obs}(%), t_{1/2}, MRT₀₋ₗₐₛₜ, MRT_{0-inf} are presented in Table G (as a non-limiting example, see FIG. 17 for illustration).

TABLE G

| | Meloxicam test | | |
|---|---|---|---|
| | formulations (in capsules) | | |
| | | Meloxicam- | |
| | Meloxicam-HPβCD | HPβCD | Mobic ® |
| | Formulation | Formulation | 7.5 mg |
| | Capsule 5.5 mg | Capsule 5.5 mg | (1 tablet, |
| Pharmacokinetic | (1 capsule, 5.5 | (2 capsules, 11.0 | 7.5 mg |
| Parameters | mg per dog) | mg per dog) | per dog) |
| Cₘₐₓ (ng/mL) | 2288 | 5428 | 2868 |
| Tₘₐₓ (h) | 1.19 | 1.06 | 3.00 |
| t_{1/2} (h) | 35.0 | 34.9 | 37.1 |
| AUC₀₋ₗₐₛₜ | 54550 | 117250 | 73225 |
| (ng/mL · hr) | | | |
| AUC_{0-inf} | 88700 | 187250 | 125750 |
| (ng/mL · hr) | | | |
| MRT₀₋ₗₐₛₜ (h) | 20.6 | 19.5 | 20.9 |
| MRT_{0-inf} (h) | 50.4 | 49.5 | 54.4 |
| AUC_{Extrap}_{_{—}}_{obs} | 37.7 | 36.6 | 41.2 |
| (%) | | | |
| Relative | 74.5 | 160 | NA** |
| Bioavailability (%)* | | | |

*relative bioavailabilities of meloxicam-HPβCD formulation capsule 5.5 mg to commercial tablets were calculated without taking the dosage into consideration.
**NA: Not applicable

Study Summary:

The meloxicam-HPβCD formulation was dosed at 5.5 mg (1 capsule per dog) and 11.0 mg (2 capsules per dog), respectively, to the group of two male and two female beagle dogs via oral administration. Mobic®, the meloxicam commercial product, was dosed at 7.5 mg (1 tablet per dog) to the same group of four beagle dogs. There was at least one week washout period between each phase. The following conclusions can be made:

The meloxicam-HPβCD formulation at both doses (1 capsule, 5.5 mg per dog, and 2 capsules, 11.0 mg per dog) show a significant faster onset, as compared to that of Mobic® tablet 7.5 mg. The Tₘₐₓ is 1.19 hours for 5.5 mg per dog, 1.06 hours for 11.0 mg per dog, while the Tₘₐₓ of Mobic® (1 tablet 7.5 mg per dog) is 3.00 hours.

The exposure (AUC and Cₘₐₓ) of the Meloxicam-HPβCD formulation (1 capsule 5.5 mg per dog) were generally comparable to that of Mobic® (1 tablet 7.5 mg per dog). The averaged relative bioavailability value of the meloxicam-HPβCD formulation capsule 5.5 mg to Mobic® tablet 7.5 mg is 74.5%.

The meloxicam-HPβCD formulation at the high dose (2 capsules, 11.0 mg per dog) showed a greater systemic exposure, as compared to those of Mobic® (1 tablet, 7.5 mg per dog). The Cₘₐₓ for meloxicam-HPβCD formulation and Mobic®, at 11 mg and 7.5 mg level were: 5428 and 2868 ng/mL, respectively; the exposure AUC₀₋ₗₐₛₜ in the same sequence, 117250 and 73225 ng/mL·hr respectively. The averaged relative bioavailability of meloxicam-HPβCD formulation (two 5.5 mg capsules, 11 mg per dog) to Mobic® (7.5 mg per dog) was 160%.

Further, as the dose of meloxicam-HPβCD formulation increases from 5.5 mg to 11.0 mg per dog, the exposure (AUC and Cₘₐₓ) of the meloxicam-HPβCD formulation increases proportionally: the Cₘₐₓ ratio of 11.0 mg over 5.5 mg is 2.37, and the AUC₀₋ₗₐₛₜ ratio of 11.0 mg over 5.5 mg is 2.15.

Clinical Study of the Meloxicam-HPβCD Formulation (Meloxicam-HPβCD Inclusion Complex) and Mobic® Under Fasting and Fed Conditions in Healthy Human Subjects.

The study objectives are to determine and compare the rates and extents of absorption of (i) a test formulation (i.e., “Meloxicam-HPβCD Formulation Capsule 5.5 mg”) with (ii) a reference Mobic® Tablet (7.5 mg). Results will be obtained after administering a single dose of the meloxicam-HPβCD formulation (5.5 mg) or Mobic® (7.5 mg) to healthy subjects under either fasting or fed conditions. Additionally, the safety and tolerability of the meloxicam-HPβCD formulation following oral administration will be analyzed. This study is a single center, randomized, open label, 4-period, 4-treatment, 4-sequence, single dose, crossover relative bioavailability study of meloxicam-HPβCD formulation and Mobic® under fasting and fed conditions in healthy subjects. The meloxicam-HPβCD formulation Capsule 5.5 mg is referred to as the test drug (T) and Mobic® Tablet 7.5 mg is referred to as the reference drug (R). A total of 16 (4×4) eligible subjects are evenly randomized to one of the four following treatment sequences according to a randomization schedule prepared prior to the start of the study, and is reproduced below (Table H):

TABLE H

| Treatment | | | | |
|---|---|---|---|---|
| Sequence | Period 1 | Period 2 | Period 3 | Period 4 |
| 1 | T1 | R1 | T2 | R2 |
| 2 | R1 | T1 | R2 | T2 |
| 3 | T2 | R2 | T1 | R1 |
| 4 | R2 | T2 | R1 | T1 |

Treatment T1: Meloxicam-HPβCD Formulation Capsule 5.5 mg; fasting conditions;
Treatment T2: Meloxicam-HPβCD Formulation Capsule 5.5 mg; fed conditions;
Treatment R1: Mobic ® Tablet 7.5 mg; fasting conditions;
Treatment R2: Mobic ® Tablet 7.5 mg; fed conditions

Each single dose administration is followed by a 7-day washout period. Subjects are dosed on the same day for Day 1 of Period 1, are crossed over to an alternate formulation and are dosed on the same day for Day 8 of Period 2, on the same day for Day 15 of Period 3, and then on the same day for Day 22 of Period 4, as shown in Table H.

During Period 1, Day 1, following an overnight fast of at least 10 hours, when either the meloxicam-HPβCD formulation or Mobic® is administered under fasting conditions, subjects receive a meloxicam-HPβCD formulation or Mobic® treatment assignment with approximately 240 mL water, where breakfast is not to be served until at least 4 hours post-dose. When the meloxicam-HPβCD formulation or Mobic® is administered under fed conditions, subjects will consume a high fat breakfast approximately 5 minutes prior to administration of the meloxicam-HPβCD formulation or Mobic®.

Serial blood samples for determination of meloxicam plasma concentration and pharmacokinetic (PK) analysis are obtained on Day 1 at time 0 (e.g., within 30 minutes pre-dose), 10 minutes, 20 minutes, 30 minutes, 45 minutes, 1 hour, 1.25 hours, 1.5 hours, 2 hours, 2.5 hours, 3 hours, 4 hours, 5 hours, 6 hours, 8 hours, 10 hours, 12 hours, 18 hours, 24 hours, 48 hours, and 72 hours post-dose. Subjects are discharged from the research facility approximately 24 hours after receiving a dose of the meloxicam-HPβCD formulation or Mobic® on Day 2 and the subjects then return for the remaining blood sample collections at approximately 0800 hours (±2 hour) on Day 3 and Day 4.

During Period 2 (i.e. Day 8), Period 3, (i.e., Day 15), and Period 4, (i.e., Day 22), following a washout period of 7 days, subjects are crossovered to an alternate reference or test formulation and the same procedures are to be performed at the same time points as noted for Period 1.

Safety assessments include monitoring adverse events (AEs), vital signs (e.g., but not limited to, blood pressure, pulse rate, respiratory rate, oral temperature, or any combination thereof), clinical laboratory findings, resting 12-lead electrocardiograms (ECGs), and physical examination findings. Vital sign assessments are performed at screening (e.g., pre-dose and daily) while each subject is sequestered in the clinic. Clinical lab testing will be performed at the screening and at the final visit for each subject. A resting 12-lead ECG will be completed at screening and at the final visit for each subject. Physical exams will be conducted at screening and at the final visit for each subject.

This study plans to enroll 16 eligible subjects.

Diagnosis and main criteria for inclusion: Healthy adult males and females between the ages of 18 and 55 years, inclusive, body mass index (BMI) between 18.5 to 32 kg/m², inclusive, healthy, as determined by no clinically significant findings from medical history, ECG, and vital signs, and who have a negative urine drug and saliva alcohol screen, and a negative pregnancy test result if female, are considered to be eligible.

Exclusion Criteria: Any clinically significant medical condition (including but not limited to renal, hepatic, gastrointestinal, cardiovascular, neurological disease), physical examination finding or clinical laboratory test result (including but not limited to: positive test results for HIV antibody, positive pregnancy tests or subject is lactating if the subject is female, positive results from a urine screen for alcohol or substances of abuse at screening or upon admission to the clinical research unit, use of any recreational drugs within the past year or a previous history of drug abuse, clinically significant ECG abnormality, etc.); Subjects with known hypersensitivity (e.g., but not limited to, anaphylactoid reactions and serious skin reactions) to meloxicam.

Investigational product, dosage and mode of administration: (i) Meloxicam-HPβCD Formulation Capsule 5.5 mg, oral formulation; (ii) Mobic® Tablet 7.5 mg, oral formulation.

Duration of treatment: The total duration of participation in the clinical study for each subject is about 30 days.

Criteria for evaluation: Safety assessments may include monitoring of adverse events (AEs), vital signs (blood pressure, pulse rate, respiratory rate and oral temperature), clinical laboratory findings, 12-lead ECGs, and physical examination findings including body weight at various time points during the study.

Pharmacokinetics: The plasma concentration time data for meloxicam is analyzed using non-compartmental methods. Actual dosing and sampling times will be used for analyses. The primary pharmacokinetics parameters of interest are: Cₘₐₓ, Tₘₐₓ, AUC₀₋ₗₐₛₜ, and AUC_{0-inf} and t_{1/2} by treatment. Additional parameters are estimated and reported as appropriate.

Relative bioavailability of the test and reference formulations is determined based on AUC₀₋ₗₐₛₜ, AUC_{0-inf} and Cₘₐₓ of meloxicam. The 90% confidence intervals (CIs) on the ratio of test to reference formulations are evaluated as to a range of 80-125%.

Statistical methods: Plasma concentration data for meloxicam are summarized using descriptive statistics (e.g., number of observations, arithmetic mean, standard deviation, median, minimum and maximum values) at each scheduled time point. PK parameters are analyzed based on scheduled sample times using non-compartmental methods and are displayed by subject and summarized by treatment. Meloxicam plasma PK profiles can be displayed graphically using untransformed and semi-log (natural logarithmic transformation) mean meloxicam concentration-time curves.

AEs (adverse events) are listed by subject and summarized by treatment. AEs are coded using the MedDRA dictionary.

This study is conducted in accordance with the guidelines of Good Clinical Practices (GCPs) including archiving of essential documents.

Summary of Study Results:

The Tₘₐₓ is decreased for meloxicam-HPβCD formulation when compared with Mobic®: under fasting conditions, there is a statistically significant difference of Tₘₐₓ between the test (“meloxicam-HPβCD formulation capsule 5.5 mg”) and the reference (Mobic® tablet 7.5 mg). The Tₘₐₓ range can be: between 0.25-3.5 hrs (e.g., but not limited to 0.25 hours, 0.5 hours, 0.75 hours, 1 hours, 1.25 hours, 1.5 hours, 1.75 hours, 2 hours, 2.25 hours, 2.5 hours, 2.75 hours, 3 hours, 3.25 hours, 3.5 hours) for the test, and between 4-6 hrs for the reference.

The absorption characteristics of meloxicam-HPβCD formulation and Mobic® are comparable (Cₘₐₓ and AUC): in both fed and fasted conditions, comparable Cₘₐₓ is achieved with the test (“Meloxicam-HPβCD Formulation Capsule 5.5 mg”) and with the reference (“Mobic® tablet 7.5 mg”).

### Equivalents

The present invention provides among other things novel methods and compositions for treating mild to moderate acute pain and/or inflammation. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein are hereby incorporated by reference in their entireties as if each individual publication or patent was specifically and individually indicated to be incorporated by reference.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

1. A method of administering a meloxicam formulation to a mammalian subject in need thereof comprising: orally administering to the subject an oral solid dosage form comprising an amorphous meloxicam-cyclodextrin inclusion complex, wherein administering the amorphous meloxicam-cyclodextrin inclusion complex results in the subject achieving a Tₘₐₓ not greater than about 3.0 hours.

2. The method of claim 1 wherein the oral solid dosage form further comprises one or more pharmaceutically acceptable excipients.

3. The method of claim 1 wherein the cyclodextrin is β-cyclodextrin.

4. The method of claim 1 wherein the cyclodextrin is a derivative of β-cyclodextrin.

5. The method of claim 1 wherein the cyclodextrin is hydroxylpropyl-β-cyclodextrin.

6. The method of claim 1 wherein the oral solid dosage form is selected from one of a capsule, a tablet, a granule powder, or a sachet.

7. The method of claim 1 for treating mild to moderate acute pain.

8. The method of claim 1 wherein the mammalian subject is a human.

9. The method of claim 1 wherein the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram.

10. The method of claim 1 wherein the amorphous meloxicam-cyclodextrin inclusion complex displays no characteristic endothermic peak of crystalline meloxicam at 250° C.-258° C. as evidenced by differential scanning calorimetry.

11. A method of administering meloxicam to a mammalian subject to manage acute pain in the subject comprising: orally administering to the subject an oral pharmaceutical formulation comprising an amorphous meloxicam-cyclodextrin inclusion complex, wherein upon administration in the subject and after an amount of meloxicam has been released from the formulation, is absorbed by the subject, and reaches the systemic circulation of the subject, a shorter T max is achieved as compared with a standard commercial formulation of meloxicam.

12. The method of claim 11 wherein the shorter Tₘₐₓ is not greater than about 75% of the Tₘₐₓ exhibited by a standard commercial formulation of meloxicam.

13. The method of claim 11 wherein the shorter Tₘₐₓ is not greater than about 50% of the Tₘₐₓ exhibited by a standard commercial formulation of meloxicam.

14. The method of claim 11 wherein the shorter Tₘₐₓ is not greater than about 25% of the Tₘₐₓ exhibited by a standard commercial formulation of meloxicam.

15. The method of claim 11 wherein upon administration in the subject and after an amount of meloxicam has been released from the formulation, is absorbed by the subject, and reaches the systemic circulation of the subject, a comparable Cₘₐₓ is achieved as compared with a standard commercial formulation of meloxicam.

16. The method of claim 11 wherein the formulation further comprises one or more pharmaceutically acceptable excipients.

17. The method of claim 11 wherein the cyclodextrin is β-cyclodextrin.

18. The method of claim 11 wherein the cyclodextrin is a derivative of β-cyclodextrin.

19. The method of claim 11 wherein the cyclodextrin is hydroxylpropyl-β-cyclodextrin.

20. The method of claim 11 wherein the formulation is selected from one of a capsule, a tablet, a sachet, or a granule powder.

21. The method of claim 11 wherein the mammalian subject is a human.

22. The method of claim 11 wherein the amorphous meloxicam-cyclodextrin inclusion complex displays typical amorphous halo and no characteristic peaks of crystalline meloxicam at 13.1, 14.9, 18.6, 25.9° at 2θ scales as evidenced by an Xray powder diffractogram.

23. The method of claim 11 wherein the amorphous meloxicam-cyclodextrin inclusion complex displays no characteristic endothermic peak of crystalline meloxicam at 250° C.-258° C. as evidenced by differential scanning calorimetry.

24. A solid pharmaceutical formulation comprising an effective amount of meloxicam, and one or more pharmaceutically acceptable excipients, the formulation comprising an amorphous meloxicam-cyclodextrin inclusion complex sufficiently designed to provide a time of meloxicam peak plasma concentration (Tmax) of not greater than about 3 hours after administration and a peak concentration (Cmax) of meloxicam which is comparable to Cmax of a standard commercial formulation of meloxicam.

25. The formulation of claim 24 wherein the pharmaceutical formulation provides effective pain control in a subject starting at about 15 minutes to 2 hours after administration and lasting for up to 24 hours after administration.

26. The formulation of claim 24 wherein the cyclodextrin is β-cyclodextrin.

27. The formulation of claim 24 wherein the cyclodextrin is a derivative of β-cyclodextrin.

28. The method of claim 24 wherein the cyclodextrin is hydroxylpropyl-β-cyclodextrin.

29. The formulation of claim 24 wherein the formulation is selected from one of a capsule, a tablet, a sachet, or a granule powder.

30. The formulation of claim 24 wherein the amorphous meloxicam-cyclodextrin inclusion complex displays typical amorphous halo and no characteristic peaks of crystalline meloxicam at 13.1, 14.9, 18.6, 25.9° at 2θ scales as evidenced by an X-ray powder diffractogram.

31. The formulation of claim 24 wherein the amorphous meloxicam-cyclodextrin inclusion complex displays no characteristic endothermic peak of crystalline meloxicam at 250° C.-258° C. as evidenced by differential scanning calorimetry.
